(19) United States
(12) Patent Application Publication
(10) Pub. No.: US 2010/0268076 A1
(43) Pub. Date: Oct. 21, 2010

(54) METHODS AND APPARATUSES FOR VASCULAR AND PROSTATE TREATMENT

(76) Inventors: Yigal Gat, Ramat-Gan (IL); Menachem Goren, Petach-Tikva (IL)

Correspondence Address: MARTIN D. MOYNIHAN d/b/a PRTSI, INC., P.O. BOX 16446, ARLINGTON, VA, 22215, US

(21) Appl. No.: 12/668,691

(22) PCT Filed: Jul. 13, 2008
(86) PCT No.: PCT/IL08/00972

§ 371 Date: Jun. 23, 2010

Related U.S. Application Data
(63) Continuation of application No. 11/826,283, filed on Jul. 13, 2007.
(60) Provisional application No. 61/064,511, filed on Mar. 10, 2008.

(51) Int. Cl. A61M 25/10; A61M 25/00; A61B 6/00

(52) U.S. Cl. 600/435; 604/508; 604/509; 600/433; 604/101.05

(57) ABSTRACT

A method of sealing a network of veins which provide drainage to the testes, comprising: (a) inserting a catheter into a main vein of said network; and (b) causing said vein to seal over at least a length, with a single application of vein irritant such that no forward movement of the catheter is required.

### RELATED APPLICATION/S

This application claims the benefit under 119(e) of 61/064,511, filed Mar. 10, 2008 by inter alia, Yigal Gat and the benefit under 120 of 11/826,283, filed Jul. 13, 2007, by inter alia, Yigal Gat. This application is also related to international patent applications, Attorney Docket Nos. 43699, Title: DIAGNOSIS AND TREATMENT OF VARICOCELE AND PROSTATE DISORDERS and 44564, Title: METHODS AND APPARATUS FOR TREATING THE PROSTATE, filed in the PCT on even date with the instant application and sharing at least inventor Yigal Gat, and which teach methods and apparatus which may be useful in conjunction with the below description. The disclosure of all of these applications is incorporated herein by reference.

### FIELD AND BACKGROUND OF THE INVENTION

The invention, in some embodiments thereof, relates to diagnosis and/or treatment of varicocele, benign prostate hyperplasia (BPH), prostate cancer and/or disorders related to the testosterone hormone. Some embodiments relate to the diagnosis and treatment to impaired testicular venous drainage.

Deterioration of the one-way valves in the internal spermatic veins, clinically manifested as varicocele, may lead to reduced drainage, or even a reflux, of venous blood into the testes.

The left internal spermatic vein (ISV) enters the left renal vein at a right angle near a potential site of compression by the superior mesenteric artery, while the right spermatic vein drains at an acute angle into the inferior vena cava (IVC). These anatomical factors, and the additional effect of gravity, promote backflow of blood in the left internal spermatic vein (more so than the right spermatic vein). Consequently, varicocele of the left ISV can be diagnosed relatively easily, and has been widely linked to male infertility in the medical literature. See, for example, Gorelick J L, Goldstein M 1993 Loss of infertility in men with varicocele, Fertility and Sterility 59, 613-616; Greenberg SH (1977) Varicocele and male fertility, Feral Steril 28(7), 699-70.

More recently, varicocele of the right ISV was recognized to play a similar role in male infertility. See, for example, Gat Y, Bachar G N, Zukerman Z and Gornish M (2004) Varicocele: a bilateral Disease, Fertil Steril 81, 424-42.

Studies over the past years demonstrated a correspondence between varicocele and serum testosterone level, though the findings did not converge to a consistent and plausible correlation. See, for example, Gat Y, Gornish M, Belenky A and Bachar G N, Elevation of serum testosterone and free testosterone after embolization of the internal spermatic vein for the treatment of varicocele in infertile men, Human Reproduction Vol. 19, No. 10 pp. 2303-2306, 2004.

Though varicocele was connected somehow with testosterone level, and testosterone is known for a long time to play a role in prostate cancer (for example, Campbell's Urology (ed-in-chief Walsh, P.) 1245-1249, 77, 2566 (Saunders Eight Edition, Philadelphia, USA, (2002)), there was no established causal correlation between varicocele and prostate disease, and, paradoxically, relatively low levels of serum testosterone were found in patients with prostate cancer (see, for example, Raivio T, Santti H, Schatzl G, Gsur A, Haidinger G, Palvimo J J, Janne O A, Madersbacher S. Reduced circulating androgen bioactivity in patients with prostate cancer. Prostate 2003; 15:194-8).

A similar paradox was also found with respect to BPH. See, for example, Roberts R O, Jacobson D J, Rhodes T, Klee G G, Leiber M M, Jacobsen S J. Serum sex hormones and measures of benign prostatic hyperplasia. Prostate. 2004 Oct. 1; 61(2):124-31).

Part of the relevant anatomy is schematically illustrated in FIG. 1 and FIG. 2. FIG. 1 schematically illustrates a typical testicular and prostate venous drainage system of a human male. One drainage path from a testis 104 comprises the pampiniform plexus 118 to the ISV 102 that leads towards the IVC 106 through one-way valves 108. Normally, the valves 108 facilitate venous blood flow upwards towards the vena cava 106, and inhibit back flow down to the testes 104.

Another drainage path comprises a sequence of pampiniform plexus 118 to the deferential vein 110, the vesicular vein 112, the internal iliac vein 114, the common iliac vein 116 towards the IVC 106. The latter path is shared by the prostate 120 drainage path from the vesicular plexus 128 towards the vesicular vein 112 and onwards.

Arteries 122 supply arterial blood to the microcirculation 124 of prostate 120 and the microcirculation 126 of testes 104.

FIG. 2 schematically illustrates typical testicular and prostate venous drainage paths in a normal left side of a human male where the arrows directions illustrate the venous blood flow as described above. Since the one-way valves 108 in the ISV 102 block back flow down to the testes 104, they isolate hydrostatic pressure from the sections between them, so that a typical pressure at the entry 142 to the left ISV 102 is about 5-6 mmHg and may be somewhat lower at entry 144 to the right ISV 130.

FIG. 3 schematically illustrates typical testicular and prostate venous drainage paths in a left side of a human male when the one-way valves in ISV 102 do not function normally, for example, due to mechanical deterioration such as weakening of valves materials, operational grinding or aging effects.

Since the one-way valves 108 in the ISVs 102, 130 do not block back flow (retrograde flow, reflux) down to the testes 104, ISVs 102, 130 form continuous columns of blood in which hydrostatic pressure develops up to approximately 31 mmHg at entry 142 to the left ISV 102 approximately 27 mmHg at entry 144 to the right ISV 130 (typically about 4-6 fold the typical pressure in ordinary conditions) when the patient is in an upright position such as standing. This excessive hydrostatic pressure, or a pressure of similar magnitude, may exist in vessels connecting to ISV 102, such as deferential vein 110 or pampiniform plexus 118, since the pressure propagates from the testicular to the prostate venous drainage systems and hydro-dynamically equilibrates between both drainage systems. The pressure may diminish as vessels are further away from entry 142 or 144, but may be still more than the normal range of about 5 mmHg.

This excessive high pressure inhibits the drainage of the venous blood from the testes 106 and the pampiniform plexus 118 up the ISV 102. Rather, the pressure pushes the testicular venous blood, rich in free testosterone (about 130 fold above serum level), towards the vesicular plexus 128 and onwards to the prostate 120, limiting drainage of venous blood from the prostate.

As the blood still circulates, venous blood from the testis is drained, at least partly, via other paths, such as the deferential vein 110, scrotal vein 128 or by-pass veins 136 that might have developed, possibly due to the excessive pressure.

The excessive pressure may produce at least some of the following effects:

(a) The venous blood that is diverted towards prostate 120 and congests and enlarges (dilates) prostate 120. The dilation of prostate 120 may be manifested, at least partially, as BPH or other prostate problems.

(b) The venous blood from the testes 106, rich in free testosterone (relative to a normal level range in the blood circulation), bathes the prostate gland cells with testosterone, effecting BPH. About 90% of the free testosterone is irreversibly converted to dehydrotestosterone (DHT), which has about five fold higher affinity for androgen receptors than free testosterone and may effects an accelerated proliferation of prostate cells. It should be noted that due to the short passage from testes 104 to prostate 120 (about 10-15 cm), only a small amount of free testosterone is bound to SHBG or albumin before bathing the prostate receptors.

(c) The excessive pressure and congestion of the prostate inhibits or reduces arterial blood from entering microcirculation 124 of the prostate and disrupts the biological balance. The excessive amounts of testosterone and DTH present in the prostate may induce an accelerated proliferation of prostate cells, and promote the development of cancer. It should be noted that the extreme concentration of free testosterone (about or over 100 fold relative to normal) in the prostate may overload the DNA hormonal feed back system, and increase the probability of mutations in the accelerated cells divisions.

(d) The excessive venous pressure inhibits or reduces arterial blood from entering the microcirculation 126 of the testes. The blood stagnates to at least some extent, and oxygenated arteriolar blood cannot flow normally into the testis, resulting in degenerative processes in the testes tissues which diminish its testosterone production.

(e) The impaired testosterone production, resulting in reduced testosterone in the blood serum, may effect aging expressions or symptoms.

The following articles relate in general to the subject of varicocele, male infertility and treatment and/or venous embolism.
Gat, Y., Zukerman, Z., Bachar, G. N., Feldberg, D., Gornish M. Adolescent varicocele: Is it a unilateral disease? Urology (2003), 62:742-746; Editorial Comment. 746 Reply by the Authors 746-747
Gat, Y., Bachar, G. N., Zukerman, Z. Gornish, M. Varicocele: a Bilateral Disease. Feral. Steril. (2004); 81:424-429. Editorial Comment in Journal of Urology, 2004 172(2) 790-791.
Gat, Y., Bachar, G., Zukerman, Z., Belenky, A., Gornish, M. Physical Examination May Miss the Diagnosis of Bilateral Varicocele. J. Urol. (2004), 172:1414-7. Editorial Comment 1239-40. 2nd Editorial Commentary and Authors' Reply, in J. Urol. 2005; June; 173(6):2208-2209
Gat, Y., Gornish, M., Belenky, A., Bachar, G. N. Elevation of serum testosterone and free testosterone after embolization of the internal spermatic vein for the treatment of varicocele in infertile men. Hum. Reprod. (2004); 19:2303-6. Editorial Comment in J Urol. (2005);173(6):2079
Gat Y, Bachar G N, Everaert K, Levinger U, Gornish M. Induction of spermatogenesis in azoospermic men after internal spermatic veins embolization for the treatment of varicocele. Hum. Reprod. (2005); 20:1013-1017. Editorial Comment in J. Urol. (2005), Nov. 174(5), 1942
Gat Y., Chakraborty, J., Zukerman, Z., Gornish, M. Varicocele, Hypoxia, and Male Infertility. Fluid mechanics analysis of the impaired testicular venous drainage system. Hum. Reprod. (2005); 20:2614-2619. Editorial Comment in J. Urol. (2006), Apr. 17(4), 1454.
Siegel Y, Gat Y, Bacher G N, Garnish M. A Proposed Anatomic Typing of the Right Internal Spermatic Vein: Importance for Percutaneous Sclerotherapy of Varicocele. Cardiovasc Intervent Radiol. 2006 March-April; 29(2):192-7.
Discussion and Author Reply in Cardiovasc Intervent Radiol. 2007 April; 30(2):348-349.
Gat Y., Gornish, M., Chakraborty, J., Navon U., Bachar G N., Ben Shlomo I. Right Varicocele and Hypoxia: Crucial factors in Male Infertility. Fluid mechanics analysis of the impaired testicular venous drainage system. Reprod Biomed Online. 2006 October; 13(4):510-5
Levinger U, Gomish M, Gat Y, Bachar G. N. Is Varicocele prevalence increasing with age? Andrologia. June, 2007, (3): 77-80.
Belenky A, Bartal G, Gat Y, Bachar G N. Uterine artery embolization: a pilot study in a rabbit model. Fertil Steril. 2005 February; 83(2):487-90.
Bachar G N, Belenky A, Greif F, Atar E, Gat Y, Itkin M, Verstanding A. Initial experience with ovarian vein embolization for the treatment of chronic pelvic pain syndrome. Isr Med Assoc J. 2004 February; 6(2):122.
Weiss D B, Gottschalk-Sabag S, Bar-On E, Zukerman Z, Gat Y, Bartoov B. [Seminiferous tubule cytological pattern in infertile, azoospermic men in diagnosis and therapy] Harefuah. 1997 May 1; 132(9):614-8, 680. Hebrew.
Gat Y., Gomish M., 2006, Technical investigation including imaging procedure for the detection of Varicocele. In: Schill, Comhaire, Hargreave (eds.). Text Book of Andrology for the Clinician. Springer Edition 2006, pp 447-453.
Gat Yigal, Varicocele: A bilateral disease. Thesis Defense for the PhD in The Medical Sciences, 19, Dec. 2006, Ghent University Hospital, Ghent, Belgium.

### SUMMARY OF THE INVENTION

An aspect of some embodiments of the invention relates to providing a method of performing scleropathy using a single shot of sclerosant. In an embodiment of the invention, performing scleropathy in a single shot is quicker than the conventional multiple shot method. In an embodiment of the invention, fluid flow in the vascular system is prevented or impeded by occlusion of at least part of one internal spermatic vein or veins that have affected undesirable hydrostatic pressures and/or back flow.

Single shot scleropathy is achieved by injecting sclerosant into the vein to be occluded using a treatment catheter while simultaneously withdrawing the treatment catheter along the part of the vein to be occluded, in an embodiment of the invention. Optionally or alternatively, some or all veins through which unwanted reflux flows are occluded using the injection/withdrawal single shot technique. Optionally or additionally, bypass veins that might have developed (for example, as a result of the hydrostatic pressure) are occluded using the injection/withdrawal single shot technique if they carry unwanted blood flow. In an embodiment of the invention, a chemical sclerosant agent is used due to its tendency to flow at least some degree down tributary and/or branch veins and occlude them, when otherwise the veins might have functioned as bypass conduits or developed into bypass conduits.

In an embodiment of the invention, a plurality of “shots” of sclerotic agent are performed, however the over all scleropathy procedure is faster than the conventional scleropathy as a result of employing fast vein access techniques and/or devices and/or using the combined one-direction motion while injecting methodology of scleropathy and/or optionally performing occlusion confirmation.

In an embodiment of the invention, the treatment catheter is provided with at least a distal balloon wherein the distal balloon at least partly hinders flow in the vasculature being treated thereby increasing the efficacy of the sclerosant being injected at the treatment site by reducing the amount of sclerosant being flushed away by fluid flow and/or preventing the sclerosant from blocking veins that should stay open. In an embodiment of the invention, providing at least a distal balloon to the treatment catheter during scleropathy is used to provide a more standardized treatment process, by removing the need to deal with fluid flow at the treatment site, and making the treatment procedure less reliant upon individual medical professional skill.

In an alternative embodiment of the invention, single shot scleropathy is achieved by occluding opposing ends of the area of the vein to be treated, thereby defining a lumen therein, and then injecting sclerosant into the lumen. In some embodiments of the invention, the opposing ends are occluded by a tandem balloon catheter provided with a distal balloon which is positioned near the inguinal region and a proximal balloon positioned near the entry orifice to the ISV. In some embodiments of the invention, a treatment catheter provided with a distal balloon (but not a proximal balloon) is used in combination with a guide catheter which is provided with an inflatable balloon, wherein the guide catheter balloon approximates the form and/or function of the proximal balloon of the tandem balloon catheter. In some embodiments of the invention, the treatment catheter is adapted to inject sclerosant between the distal and proximal balloons. Optionally, the treatment catheter is provided with a side hole port for injection of sclerosant and/or aspiration of the lumen. In some embodiments of the invention, at least one port is located at the distal tip of the treatment catheter. In a telescoping embodiment of the treatment catheter, the catheter stays in place while a balloon is moved towards the proximal end of the catheter to push aside valves and/or pull out sclerosant.

In an alternative embodiment of the invention, single shot occlusion of vasculature is achieved alone or with the assistance of other irritants, for example a brush and/or wire which mechanically irritate the vascular wall causing swelling and therefore, occlusion.

An aspect of some embodiments of the invention relates to a distal balloon fitted treatment catheter adapted to perform scleropathy. In an embodiment of the invention, the distal balloon is selectively inflatable and deflatable by using a side hole port with a fluid connection between a balloon inflation/deflation lumen located within the treatment catheter and the inside area of the balloon. In an embodiment of the invention, the distal balloon is adapted to inflate sufficiently to substantially hinder fluid flow through the vasculature in which the treatment catheter is located. Optionally, the vasculature is an ISV. In some embodiments of the invention, the treatment catheter is adapted for use in a single shot scleropathy procedure wherein the treatment catheter can inject sclerosant and be withdrawn with the balloon inflated simultaneously. In some embodiments of the invention, the treatment catheter is adapted for use in small vein spaces, for example those found around venous plexuses like the pampiniform plexus.

The treatment catheter is also provided with a contrast agent and/or sclerosant lumen through which contrast agent and/or sclerosant are fed through the treatment catheter to a treatment site within the vasculature, in some embodiments of the invention. In an embodiment of the invention, the treatment catheter is adapted to inject contrast agent and/or sclerosant on the proximal side of the distal balloon (i.e. downstream from the balloon). Optionally, aspiration/evacuation of the treatment site is performed using the contrast agent and/or sclerosant lumen by applying suction to the lumen.

In an embodiment of the invention, the treatment catheter is provided with a guide wire lumen through which a guide wire passes when the treatment catheter is being moved to the treatment site. Optionally, the guide wire is retracted from the lumen when the treatment catheter reaches its proper position for treatment.

In some embodiments of the invention, the distal balloon fitted treatment catheter is adapted to perform a valve breakthrough procedure by inflating the balloon just downstream of the valve creating a vacuum effect, opening the valve, and enabling the transit of the treatment catheter therethrough. In an embodiment of the invention, the balloon is deflated after the valve has been passed through.

In some embodiments of the invention, the distal balloon fitted treatment catheter is also provided with a proximal balloon, wherein the distal and proximal balloons define a space therebetween. In an embodiment of the invention, the treatment catheter is adapted to inject sclerosant between the distal and proximal balloons via an injection side hole port. In an embodiment of the invention, the proximal balloon is selectively inflatable and deflatable.

An aspect of some embodiments of the invention relates to a tandem treatment catheter adapted to perform single shot scleropathy with a proximal and a distal balloon without moving the treatment catheter during injection of the sclerotic agent. In an embodiment of the invention, the proximal and distal balloons are selectively inflatable and deflatable by using side hole ports with a fluid connection between each balloon's inflation/deflation lumen located within the treatment catheter and the inside area of the balloons. In an embodiment of the invention, the distal balloons are adapted to inflate sufficiently to substantially hinder fluid flow through the vasculature in which the treatment catheter is located. Optionally, the vasculature is an ISV. In an embodiment of the invention, when inflated the distal and proximal balloons define a lumen or space therebetween. In an embodiment of the invention, the treatment catheter is adapted to inject sclerosant between the distal and proximal balloons via an injection side hole port.

In an embodiment of the invention, the tandem treatment catheter is provided with a contrast agent and/or sclerosant transporting lumen. Optionally, the space between the balloons is aspirated or evacuated using the contrast agent and/or sclerosant transporting lumen by applying suction. In some embodiments of the invention, each balloon is provided with its own inflation/deflation lumen whereby each balloon can be individually inflated and/or deflated. Optionally, the tandem treatment catheter is provided with a guide wire lumen through which a guide wire passes when the treatment catheter is being moved to the treatment site. Optionally, the guide wire is retracted from the lumen when the tandem treatment catheter reaches its proper position for treatment.

An aspect of some embodiments of the invention relates to a fast vein access procedure and/or devices adapted to provide fast vein access. In some embodiments of the invention, guide catheters which are pre-shaped for ISV access are provided. In some embodiments of the invention, a guide catheter adapted for providing access to a right ISV is provided with an engagement tip wherein the engagement tip has a protrusion that engages a dip at the intersection of IVC and the ISV. This dip is at the place where the valve of the ISV might be. In an embodiment of the invention, the dip is found by inserting and wiggling around catheter. Once the dip is found, access to the ISV is gained by applying vacuum, pushing through the valve and/or deforming ISV so that valve is open.

An aspect of some embodiments of the invention relates at least one to guide catheter provided with a selectively inflatable balloon at the distal end (i.e. tip) of the catheter. In an embodiment of the invention, the balloon is adapted to substantially hinder fluid flow through the vasculature in which the balloon is located. Optionally, the vasculature is an ISV. In some embodiments of the invention, the balloon of the guide catheter is used in combination with a distal balloon of a treatment catheter, the two balloons defining a lumen therebetween. In an embodiment of the invention, the lumen defined therebetween is a scleropathy treatment site. In an embodiment of the invention, the guide catheter is adapted to provide a plurality of lumens for at least one of a guide wire, a treatment catheter, and/or inflation/deflation of the balloon.

In some embodiments of the invention, the at least one guide catheter is preformed for interfacing with a specific anatomical feature. Optionally, the anatomical feature is a right ISV. Optionally, the anatomical feature is a left ISV. Optionally, the anatomical feature is a right renal vein. Optionally, the anatomical feature is a left renal vein.

An aspect of some embodiments of the invention relates to a method of gaining expedited vein access using pre-shaped guide catheters tailored for specific veins and/or vein orifices and/or approach angles to the veins and/or combining the pre-shaped guide catheters with an expedited valve breakthrough procedure. In an embodiment of the invention, a distal balloon fitted treatment catheter is advanced through the guide catheter towards and into the vein to be treated. In an embodiment of the invention, the expedited valve breakthrough procedure is achieved by inflating the balloon on the treatment catheter just downstream of a valve to be bypassed creating a vacuum effect, opening the valve, and enabling the transit of the treatment catheter therethrough. In an embodiment of the invention, the balloon is deflated after the valve has been passed through.

An aspect of some embodiments of the invention relates to irritation devices for instigating at least temporary vessel occlusion using mechanical stimulation of the vessel wall.

In an embodiment of the invention, an irritation device is adapted to instigate occlusion of a vessel by providing a brush structure to a distal end of the irritation device. In an embodiment of the invention, the brush structure is comprised of a plurality of short bristles extending along a length of the device from the distal end and around the circumference of the device, similar to a pipe cleaner. In an embodiment of the invention, the bristles are flexible. Optionally, the bristles are made of a biocompatible material such as nylon. In an embodiment of the invention, the bristles are adapted to abrade the inner surface of a vessel wall. In an embodiment of the invention, the brush irritation device is adapted to be inserted into and/or deployed from a delivery catheter, wherein the delivery catheter provides a sheltered housing for the irritation device which prevents irritation of the vessel wall while the irritation device is inside it. In an embodiment of the invention, deployment from the delivery catheter and withdrawal of the brush irritation device along the section of vessel to be occluded is performed as a single action.

In an embodiment of the invention, an irritation device is adapted to instigate occlusion of a vessel using an arm assembly attached to a distal end of the irritation device. In an embodiment of the invention, a plurality of arms extend radially and/or longitudinally (i.e. along the axis of the device) out from the distal end. Optionally, the arms are gently curved. In some embodiments of the invention, the arms are equally spaced from each other (in a radial sense). Optionally, the anus are made of a biocompatible metal material. Optionally, the arms are made of a biocompatible plastic material. In an embodiment of the invention, the arms are adapted to prevent puncturing a wall of the vessel. For example, the ends of the arms are rounded. In some embodiments of the invention, the arms of the device are adapted to extend out from the device to the vessel wall and with enough tension to abrade the wall's inner surface. In an embodiment of the invention, the arm assembly irritation device is adapted to be inserted into and/or deployed from a delivery catheter, wherein the delivery catheter provides a sheltered housing for the irritation device which prevents irritation of the vessel wall while the irritation device is inside it. In an embodiment of the invention, deployment from the delivery catheter and withdrawal of the arm assembly irritation device along the section of vessel to be occluded is performed as a single action.

There is thus provided in accordance with an exemplary embodiment of the invention, a method of sealing a network of veins which provide drainage to the testes, comprising: (a) inserting a treatment catheter into a main vein of said network; and, (b) causing said vein to seal over at least a length with a single application of vein irritant such that no forward movement of the treatment catheter is required.

In an embodiment of the invention, the single application comprises pulling back said catheter while applying a sclerotic agent. In some embodiments of the invention, a distal balloon provided to the catheter is inflated prior to the application of vein irritant. Optionally, the efficacy of the vein irritant is increased by the distal balloon at least partly hindering flow in the main vein.

In an embodiment of the invention, the single application comprises injecting a sclerotic agent into a lumen which proscribes the length to be sealed. Optionally, a distal balloon and a proximal balloon are inflated, one on each end of the length, thus defining the lumen. Optionally, the distal balloon and the proximal balloon are inflated using the treatment catheter. Optionally, the distal balloon is inflated using the treatment catheter and the proximal balloon is inflated using a guide catheter.

In an embodiment of the invention, the catheter is used to map the network of veins by injecting a contrast agent into the network from the catheter. Optionally, the length is chosen for sealing based on the map of the network of veins.

In an embodiment of the invention, sealing the network is used for treating at least one of BPH, varicocele, cancer, and the reflux of venous blood from the testes to the prostate.

In an embodiment of the invention, the sclerotic agent is a chemical agent.

There is further provided in accordance with an exemplary embodiment of the invention, a treatment catheter adapted for performing scleropathy, comprising: a shaft defining a longitudinal axis of the catheter; a selectively inflatable and deflatable distal balloon located on the shaft; a side hole port in fluid communication with the distal balloon and a balloon inflation/deflation lumen for selectively inflating and deflating the distal balloon; a side hole port in fluid communication with a contrast agent and sclerosant lumen and a lumen of a vein being treated, wherein the side hole port is adapted to inject the contrast agent and the sclerosant downstream in the lumen from the distal balloon.

In an embodiment of the invention, aspiration of the lumen of the vein being treated is achieved by applying suction through the contrast agent and sclerosant lumen.

In an embodiment of the invention, the treatment catheter further comprises a guide wire lumen adapted for transit of a guide wire therethrough.

In an embodiment of the invention, the treatment catheter further comprises a selectively inflatable and deflatable proximal balloon, wherein the distal and proximal balloons define the lumen of the vein being treated therebetween. Optionally, the treatment catheter is adapted to inject sclerosant into the lumen of the vein being treated between the proximal and distal balloons.

In an embodiment of the invention, the treatment catheter further comprises an engagement tip located at a distal end of the shaft and adapted for engaging an orifice of an ISV.

There is further provided in accordance with an exemplary embodiment of the invention, a treatment catheter adapted for performing scleropathy, comprising: a selectively inflatable and deflatable proximal balloon; and, a selectively inflatable and deflatable distal balloon, wherein the proximal balloon and the distal balloon proscribe a lumen therebetween to be treated by scleropathy.

In an embodiment of the invention, the treatment catheter further comprises dedicated inflation/deflation lumens for the proximal balloon and the distal balloon. In an embodiment of the invention, the treatment catheter further comprises side hole ports in fluid communication with each dedicated inflation/deflation lumen and the proximal balloon and the distal balloon. Optionally, at least one balloon is adapted to substantially restrict flow in the lumen. Optionally, the lumen is of an ISV.

In an embodiment of the invention, the treatment catheter is adapted to inject sclerosant into the lumen between the proximal and distal balloons.

In an embodiment of the invention, the treatment catheter further comprises a guide wire lumen adapted for transit of a guide wire therethrough.

In an embodiment of the invention, the treatment catheter further comprises a contrast agent and sclerosant transporting lumen. Optionally, aspiration of the lumen of the vein being treated by scleropathy is achieved by applying suction through the contrast agent and sclerosant lumen.

In an embodiment of the invention, the treatment catheter further comprises an engagement tip located at a distal end of the shaft and adapted for engaging an orifice of an ISV.

There is further provided in accordance with an exemplary embodiment of the invention, a guide catheter with a preconfigured shape, comprising: an elongate tube defining a lumen suitable to receive, at a proximal end, a treatment catheter therethrough; and, at least one projection at a tip of the tube and adapted to engage a recess of a right ISV valve.

In an embodiment of the invention, the guide catheter further comprises a selectively inflatable and deflatable balloon located at a distal end of the guide catheter. Optionally, the balloon is the at least one projection at the tip.

In an embodiment of the invention, the tube defines a first curve of at least 170 degrees.

In an embodiment of the invention, the guide catheter includes a second curve in the opposite direction of the first curve wherein the second curve is adapted to approximate the angle of incidence of the right ISV to the IVC and is located distally of the first curve.

Optionally, the catheter is between 600 mm and 700 mm long.

In an embodiment of the invention, the distal balloon is adapted to substantially occlude vasculature in which the balloon is located, thereby hindering fluid flow. Optionally, the vasculature is an ISV.

In an embodiment of the invention, the guide catheter is provided with a lumen for at least one of a guide wire or air for selectively inflating and deflating the balloon.

In an embodiment of the invention, the guide catheter further comprises an engagement tip located at the distal end of the elongate tube and adapted for engaging an orifice of an ISV.

In an embodiment of the invention, the selectively inflatable and deflatable balloon is at least a part of the engagement tip.

Optionally, the guide catheter is flexible.

There is further provided in accordance with an exemplary embodiment of the invention, a guide catheter with a preconfigured shape, comprising: an elongate tube defining a lumen suitable to receive, at a proximal end, a treatment catheter therethrough; and, wherein the elongate tube's shape is adapted to be positioned within a left renal vein and a left ISV. In an embodiment of the invention, the elongate tube has a first curve of 70-130 degrees approximating the angle of entry to the IVC by the left renal vein and a second curve of approximately 100-125 degrees approximating the angle of entry of the left ISV to the left renal vein. Optionally, the guide catheter is 650 mm in length.

In an embodiment of the invention, the guide catheter further comprises a selectively inflatable and deflatable balloon located at a distal end of the guide catheter. Optionally, the balloon is the at least one projection at the tip.

In an embodiment of the invention, the distal balloon is adapted to substantially occlude vasculature in which the balloon is located, thereby hindering fluid flow. Optionally, the vasculature is an ISV.

In an embodiment of the invention, the guide catheter is provided with a lumen for at least one of a guide wire or air for selectively inflating and deflating the balloon.

There is further provided in accordance with an exemplary embodiment of the invention, a method for gaining expedited vein access to veins which drain the testes, comprising: inserting a pre-shaped guide catheter into an access point; maneuvering the guide catheter into position opposite an anatomical feature of interest; interfacing the guide catheter with the anatomical feature; inserting a treatment catheter into the guide catheter and advancing the treatment catheter to a first valve of the anatomical feature; inflating a distal balloon of the treatment catheter just downstream of the first valve thereby creating a vacuum effect on the valve causing the valve to open; advancing the treatment catheter past the valve; and, deflating the balloon after traversal of the valve.

In an embodiment of the invention, the method for gaining expedited vein access to veins which drain the testes further comprises a advancing the treatment catheter towards a treatment site.

In an embodiment of the invention, the method for gaining expedited vein access to veins which drain the testes further comprises repeating inflating, advancing the treatment catheter past a valve, deflating and advancing the treatment catheter towards a treatment site until the treatment site is reached.

There is further provided in accordance with an exemplary embodiment of the invention, an irritation device, comprising: a shaft, and a brush structure at a distal end of the shaft, wherein the brush structure is adapted to irritate the inner wall of a blood vessel.

In an embodiment of the invention, the brush structure is comprised of a plurality of short bristles. Optionally, the brush structure is constructed of nylon. Optionally, the brush structure is flexible.

In an embodiment of the invention, the irritation device is adapted to be inserted into and deployed from a delivery catheter.

There is further provided in accordance with an exemplary embodiment of the invention, an irritation device, comprising: a shaft; and, an arm assembly at the distal end of the shaft, wherein the arm assembly is adapted to irritate the inner wall of a blood vessel.

In an embodiment of the invention, the arm assembly is comprised of a plurality of arms. Optionally, the plurality of arms extends at least one of radially and longitudinally from the distal end of the shaft. Optionally, the arms are gently curved. Optionally, the arms are equally spaced from each other in a radial sense.

In an embodiment of the invention, the arm assembly is constructed of metal.

In an embodiment of the invention, the arm assembly is constructed of plastic.

In an embodiment of the invention, the arms are adapted to prevent puncturing the wall of the blood vessel.

In an embodiment of the invention, the irritation device is adapted to be inserted into and deployed from a delivery catheter.

There is further provided in accordance with an exemplary embodiment of the invention, a method for treating varicocele, comprising: (a) determining that a patient has incompetent valves in a venus plexus; (b) inserting a catheter into the venous plexus; (c) sealing the venous plexus using a single injection of sclerosant which reduces venous pressure on a testis using the catheter.

In an embodiment of the invention, the method further comprises, (d) confirming sealing of the venous plexus by injecting a contrast agent into the venous plexus and observing fluid flow therein.

Optionally, the network of veins is sealed in 20-30 minutes.

Optionally, the network of veins is sealed in 30-45 minutes.

Optionally, the network of veins is sealed in under an hour.

There is further provided in accordance with an exemplary embodiment of the invention, a method of sealing a network of veins which provide drainage to the testes, comprising: (a) inserting a treatment catheter into a main vein of said network; and, (b) causing said vein to seal over at least a length using a plurality of applications of vein irritant without changing direction of a withdrawal motion of the treatment catheter.

In an embodiment of the invention, the method further comprises stopping after a valve in the vein and just prior to at least one of the plurality of applications. Optionally, the withdrawal motion instigates a flattening of the valve as the catheter passes through the valve.

There is further provided in accordance with an exemplary embodiment of the invention, a kit for performing scleropathy, comprising: at least one treatment catheter; and, at least one pre-shaped guide catheter adapted for passage therethrough of the treatment catheter.

In an embodiment of the invention, the kit further comprises a vein sheath.

In an embodiment of the invention, the kit further comprises sclerosant for use with the treatment catheter.

In an embodiment of the invention, the kit further comprises a contrast, material.

In an embodiment of the invention, the kit further comprises instructions for using at least one of the treatment catheter, the pre-shaped guide catheter, the vein sheath, the sclerosant or the contrast material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. Illustrations and labels of the left side of the human anatomy apply also to the right side, unless specifically indicated.

In the drawings:

FIG. 1 schematically illustrates a typical testicular and prostate venous drainage system of a human male;

FIG. 2 schematically illustrates typical testicular and prostate venous drainage paths in a normal left side of a human male;

FIG. 3 schematically illustrates typical testicular and prostate venous drainage paths in a left side of a human male when the one-way valves in the internal spermatic vein do not function;

FIG. 4A is a flowchart showing a single shot scleropathy procedure, in accordance with an exemplary embodiment of the invention;

FIG. 4B is a flowchart showing a single shot scleropathy procedure using a dual occlusion configuration, in accordance with an exemplary embodiment of the invention;

FIGS. 5A-5B are illustrations of left and right fast access guide catheters, in accordance with exemplary embodiments of the invention;

FIGS. 5C-5D are illustrations of left and right fast access guide catheters with balloon tips, in accordance with exemplary embodiments of the invention;

FIGS. 6A-6B are illustrations of left and right fast access guide catheters in vivo, in accordance with exemplary embodiments of the invention;

FIG. 7 is a flowchart showing an expedited vein access procedure, in accordance with an exemplary embodiment of the invention;

FIG. 8A schematically illustrates a treatment catheter including an optional distal balloon and multiple input/output ports, in accordance with an embodiment of the invention;

FIG. 8B is a cross sectional view of the treatment catheter of FIG. 8A, in accordance with an exemplary embodiment of the invention;

FIG. 8C is a perspective view of a distal tip of the treatment catheter of FIG. 8A with an optional distal balloon attached, in accordance with an exemplary embodiment of the invention;

FIG. 8D schematically illustrates a treatment catheter, in accordance with an exemplary embodiment of the invention;

FIG. 8E is a cross sectional view of the treatment catheter of FIG. 8D, in accordance with an exemplary embodiment of the invention;

FIG. 9A schematically illustrates a tandem balloon treatment catheter, in accordance with an exemplary embodiment of the invention;

FIG. 9B is a cross sectional view of the tandem balloon treatment catheter of FIG. 9A at a non-balloon segment, in accordance with an exemplary embodiment of the invention;

FIG. 9C is a cross sectional view of the tandem balloon treatment catheter of FIG. 9A at a balloon segment, in accordance with an exemplary embodiment of the invention;

FIG. 10A is a side view of a brush irritation device, in accordance with en exemplary embodiment of the invention;

FIG. 10B is a side view of the brush irritation device of FIG. 10A in a delivery catheter, in accordance with en exemplary embodiment of the invention;

FIG. 10C is a side view of the brush irritation device of FIG. 10A deployed from a delivery catheter, in accordance with an exemplary embodiment of the invention;

FIG. 10D is a cross sectional view of the brush irritation device of FIG. 10A across the brush section and including the delivery catheter, in accordance with an exemplary embodiment of the invention;

FIG. 11A is a side view of an arm assembly irritation device, in accordance with an exemplary embodiment of the invention;

FIG. 11B is a cross sectional view of the arm assembly, in accordance with an exemplary embodiment of the invention;

FIG. 11C is a side view of the arm assembly irritation device of FIG. 11A in a delivery catheter, in accordance with an exemplary embodiment of the invention;

FIG. 11D is a side view of a delivery catheter and the arm assembly irritation device of FIG. 11A being inserted into a vessel to be treated, in accordance with an exemplary embodiment of the invention; and,

FIG. 11E is side view of the arm assembly irritation device of FIG. 11A deployed from a delivery catheter, in accordance with an exemplary embodiment of the invention.

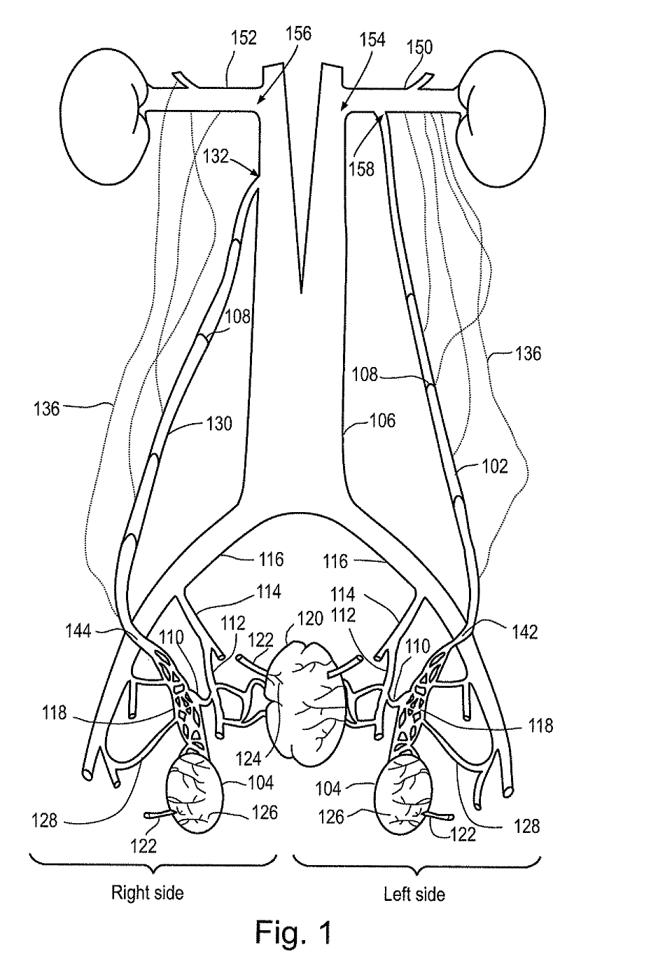

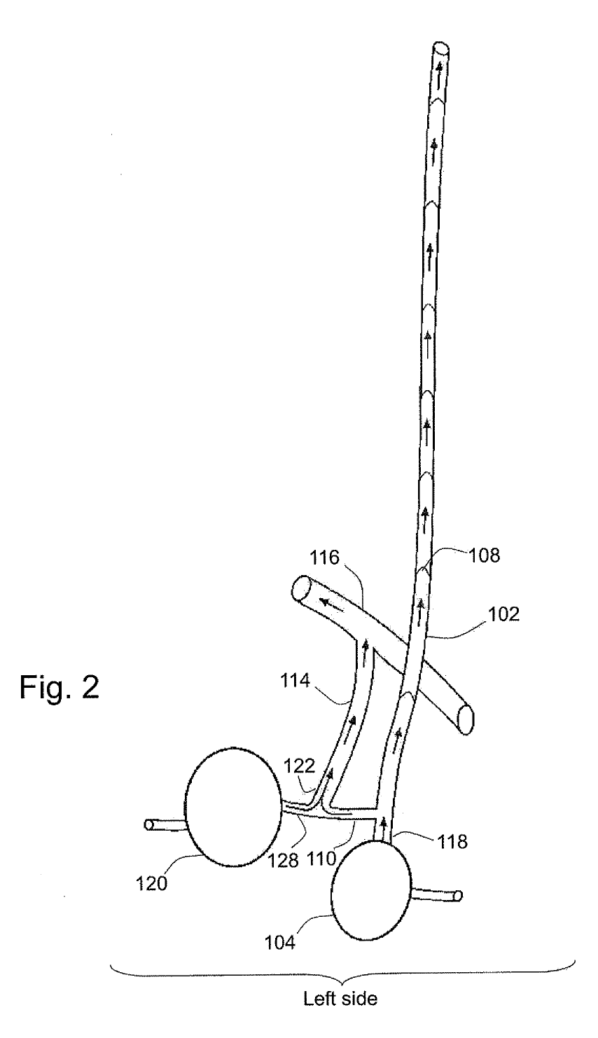

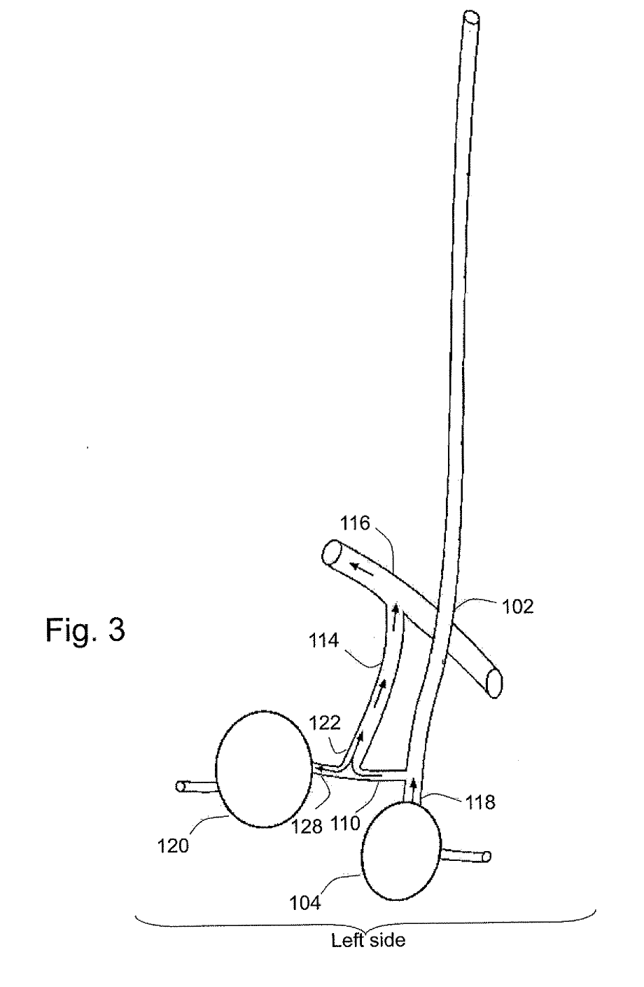

### DESCRIPTION OF EXEMPLARY EMBODIMENTS OF THE INVENTION

In the specifications and claims the terms ‘left side’ and ‘right side’ refer to the conventional anatomical terminology (e.g. the heart, stomach and spleen are on the left side of most human beings). In the specifications and claims the term “drainage” refers to a flow of venous blood via venous vessels towards and into the vena cava, and the terms “reflux” and “backflow” are used synonymously for flow in the opposite direction of “drainage”. It should be noted that anatomical features can vary from person to person and that methods and/or devices described herein are intended for use in a wide variety of anatomical configurations.

In some exemplary embodiments of the invention, one or more of the adverse states and effects described above may be avoided, delayed, alleviated and/or repaired, at least to some degree, by reducing or eliminating the excessive pressure. Reducing the pressure reduces or eliminates varicocele, BPH, infertility, some forms of cancer and/or the back flow (reflux) of venous blood, rich in testosterone (relative to normal levels in the blood circulation), from the testes to the prostate.

The headings herein are not limiting and are intended for clarity only.

### Single Shot Scleropathy Procedure

As indicated in U.S. patent application Ser. No. 11/826,283 and U.S. Patent Application No. 61/064,511, the reflux can be prevented or impeded by occlusion (e.g. using embolization or sclerosis) of the left ISV 102 and/or the right ISV 130 that has affected the excessive hydrostatic pressures. Optionally and/or additionally, some or all veins through which the reflux flows, such as the deferential vein 110 and the pampiniform plexus 118, are occluded. Optionally or additionally, bypass veins 136 that might have developed are occluded too. In an embodiment of the invention, left ISV 102 is treated before right ISV 130. It should be noted however, that right ISV 130 could be treated first instead.

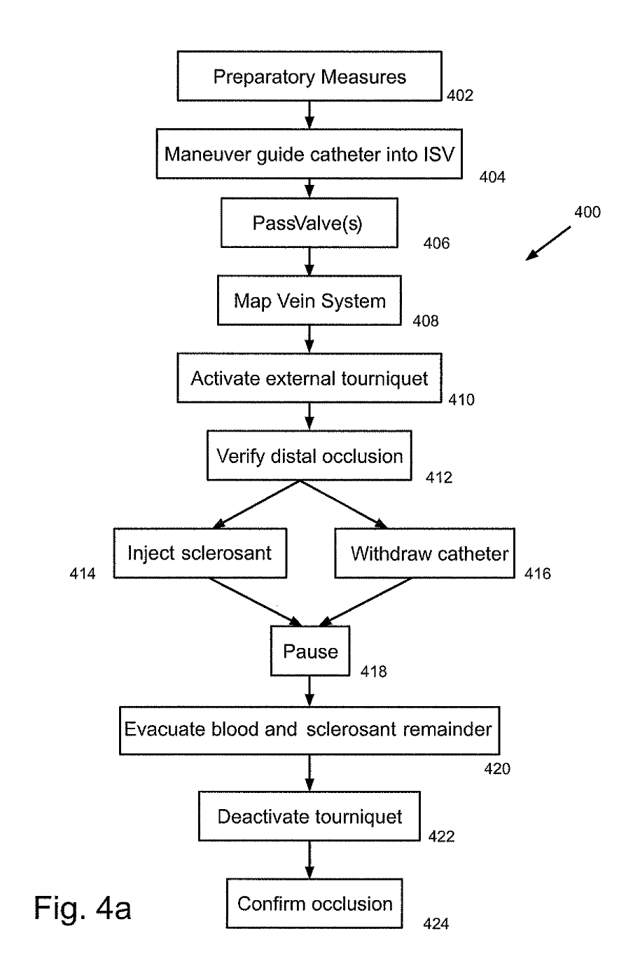

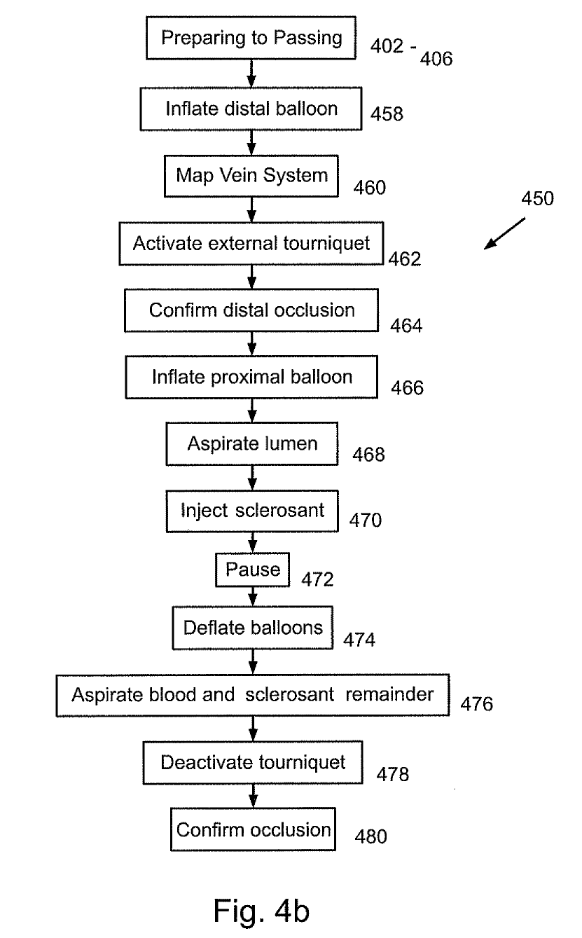

Referring to FIG. 4A, a flowchart 400 is shown illustrating a single shot scleropathy procedure, in accordance with an exemplary embodiment of the invention. In an exemplary embodiment of the invention, “single shot” means that occlusion of a length of a vein that would conventionally require multiple discrete injections of sclerosant accompanied with multiple discrete movements of the treatment catheter is performed instead by using a single injection accompanied with a single, continuous motion of the treatment catheter (or no movement of the treatment catheter at all, as described with respect to FIG. 4B).

An advantage of using the single shot method is a reduction in the time it normally takes to render scleropathy treatment for such maladies as varicocele, BPH, reflux of blood to the prostate, etc. In an embodiment of the invention, the single shot procedure is performed in 20-30 minutes. In some embodiments of the invention, the single shot procedure is performed in 30-45 minutes. Optionally, the single shot procedure is performed in under an hour. It should be noted that each patient's needs are different and that variations in needs and/or anatomy and/or medical condition requires varying levels of time commitment from the attending medical professional. In some embodiments of the invention, the single shot procedure is combined with a fast vein access procedure, described below, to reduce the overall amount of time it takes to perform scleropathy.

The amount of sclerosant injected is matched to the movement speed of the injection catheter to maximize occlusion effectiveness, in some embodiments of the invention. Assuming diameters ranging from 1 mm-4 mm, the vessels to be treated may have a volume of 0.3 ml of sclerosant injected for a 1 cm segment of a 2 mm vein to 0.7 ml of sclerosant injected for a 1 cm segment of a 3 mm vein to 1.3 ml for a 1 cm segment of a 4 mm vein, in accordance with some exemplary embodiments of the invention. In instances of treating varicocele, it is possible that diameters of vessels being treated might be larger than 1 mm-4 mm and that described methods and/or devices herein are adaptable for use in these larger diameter spaces. It should be understood that some or all of the actions described herein (e.g. insertion/retraction of devices, inflation/deflation of balloons, injection/aspiration of materials) could be performed by a controller either by command of an operator of the controller and/or automatically.

In an embodiment of the invention, preparatory measures are taken (402) to prepare the patient for entry of treatment devices into a target ISV 102, 130. Acquiring percutaneous access of the femoral vein is desired, in an embodiment of the invention. In some embodiments of the invention, the right femoral vein is used. Optionally, the left femoral vein is used. The localization of the femoral vein puncture site is optionally confirmed by physical examination: at or below a line from the anterior superior iliac spine to the symphysis pubis. Local anesthesia is optionally applied around the puncture site and a Valsalva maneuver is optionally performed to distend the femoral vein while the vein is punctured with an optionally sheathed femoral puncture needle. Optionally, the femoral puncture needle is at least 18 gauge.

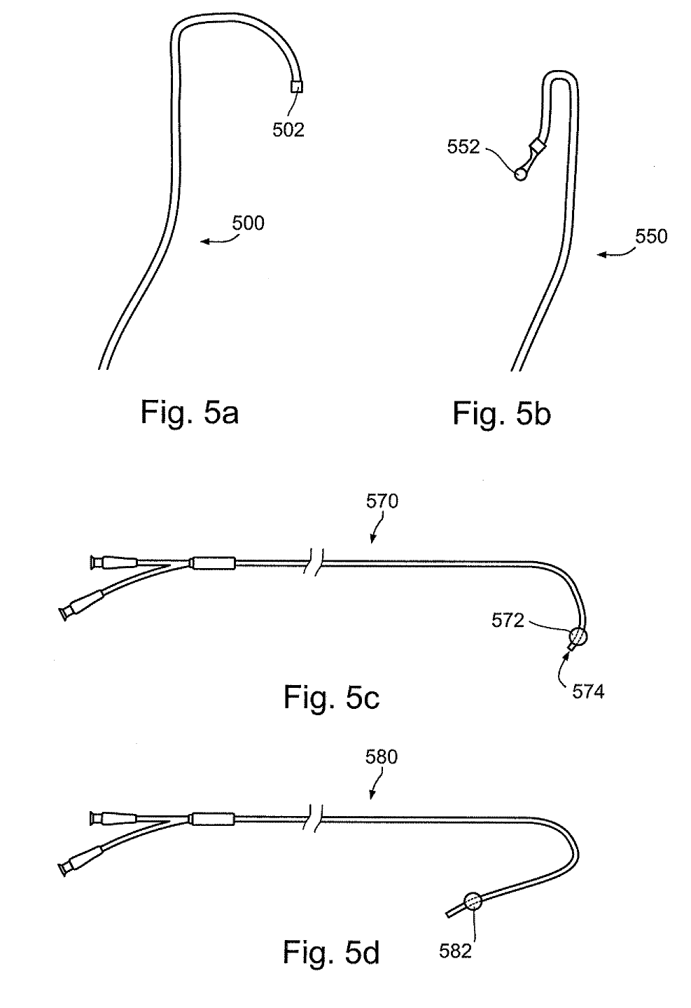

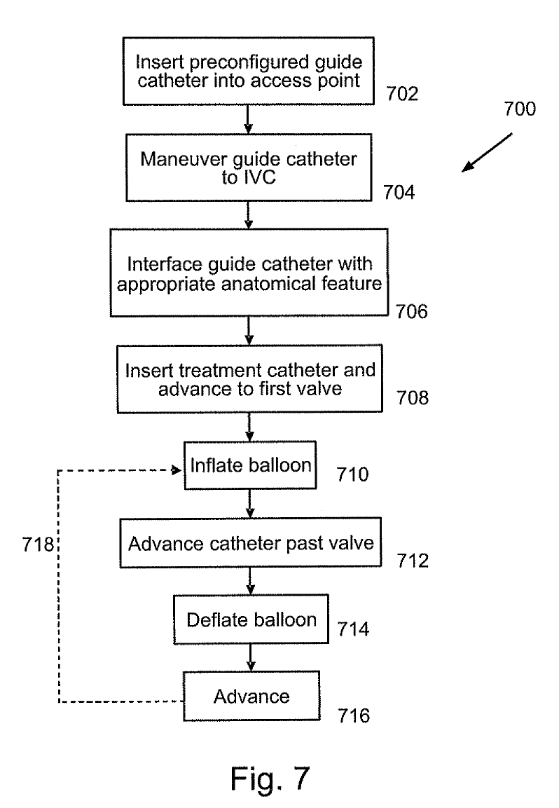

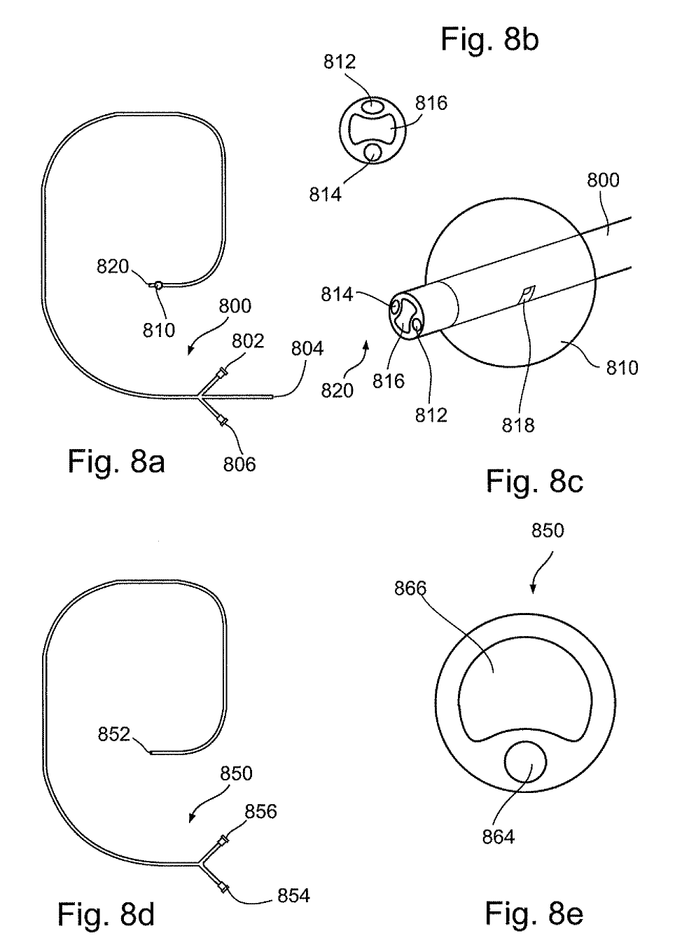

In some embodiments of the invention, a coaxial system is used, with a femoral vein sheath and at least one of two pre-shaped guiding catheters 500, 550, one catheter 500 shaped for entry into the left ISV 102 and one catheter 550 shaped for entry into the right ISV 130, as shown and described in more detail with respect to FIGS. 5A-5D and 7. Optionally, the procedure is performed with a 6 French sheath and 6 French pre-shaped guiding catheters 500, 550 through which a treatment catheter 850, shown and described in more detail with respect to FIGS. 8D-8E, is placed. Optionally, treatment catheter 850 is 3 F. A smaller or larger sheath and/or guiding catheters can be used in some embodiments of the invention provided they allow the passage of the treatment catheter 850 therethrough. Optionally, a smaller or larger treatment catheter is used provided it fits through any sheaths and/or guide catheters that are used and/or are sized for treatment of and/or transit through the patient's anatomical features.

The venous puncture is optionally performed as a two wall puncture to allow additional local anesthesia to be placed deep into the vein. In an embodiment of the invention, a guide wire is placed through the needle into the vein and advanced into the pelvis. Optionally, the guide wire is a 0.014-0.018 inch guide wire. In some embodiments of the invention, the guide wire is provided with a variably curved and/or soft and/or flexible engagement tip that is adapted to be used to directly engage the orifice of the ISV. Additionally, optionally and/or alternatively, the treatment catheter is provided with a variably curved and/or soft and/or flexible engagement tip that is adapted to be used to directly engage the orifice of the ISV. In an embodiment of the invention, the engagement tip is maneuvered into the orifice by the attending medical professional using skill and/or feel and/or imaging. The needle or sheath is withdrawn around the guide wire while compressing the puncture site. In an embodiment of the invention, the sheath is temporarily placed over the guide wire with its external port at the groin while a side port of the sheath is flushed with heparinized saline solution. Optionally, this is repeated throughout the procedure at regular intervals. Optionally, the intervals are 5-10 minute intervals.

In an embodiment of the invention, at least one of the pre-shaped guiding catheters 500, 550 is advanced into the IVC 106 over the guide wire. Depending on which ISV 102, 130 is being targeted and/or the particular anatomy of the patient, slightly different procedures are optionally followed in order to navigate guiding catheter 500, 550 to the appropriate ISV 102, 130. In an embodiment of the invention, treatment catheter 850 is optionally provided with a guide wire lumen 864, shown in FIG. 8E, through which the guide wire passes if treatment catheter 850 is being advanced by following the guide wire. In an embodiment of the invention, the guide wire is fed into treatment catheter 850 using an inlet port 854.

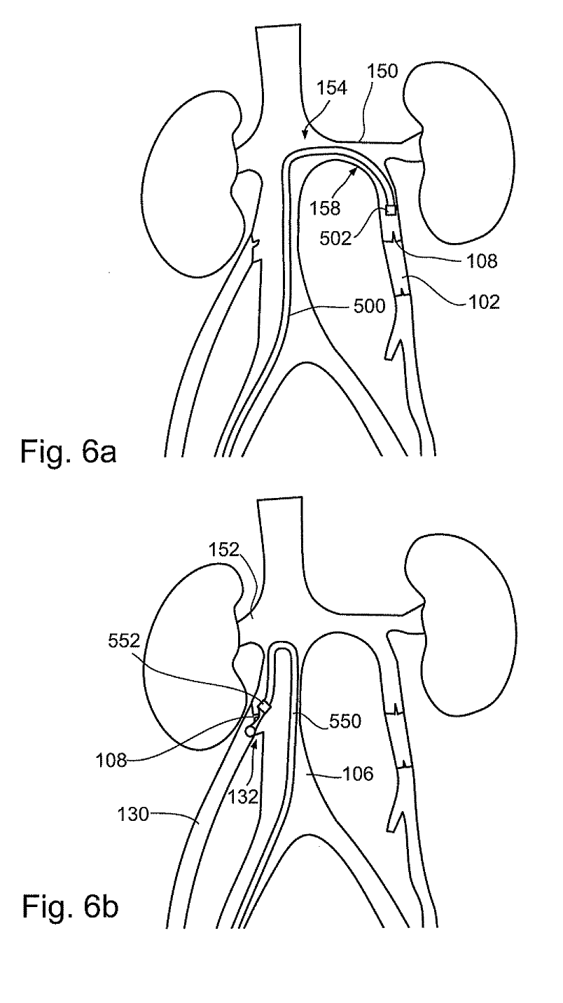

In an embodiment of the invention, guiding catheter 500 is maneuvered (404) into the orifice 154 of the left renal vein 150 (in the case of treating ISV 102) under fluoroscopic control, as shown in more detail in FIG. 6A. In about 5% of cases, the junction of the left renal vein with IVC 106 is an obtuse angle, heading caudally or cephalad from the kidney. In such instances, the ISV 102 orifice 158 joins the left renal vein 150 at an acute angle, making it difficult to enter and/or difficult to find from the upwards slanting renal vein 150. In another 2%-3% of cases, the ISV 102 joins the renal vein 150 together with or just below a paraaortic vein. In these cases, guiding catheter 500 needs to be maneuvered so that a tip 502 overlies the orifice 158, and, using a combination of suspended respirations, tilting of the fluoroscopic table, and gentle, but firm rotation of the catheter 500, the orifice 158 of the ISV 102 is engaged, in accordance with an exemplary embodiment of the invention. In some embodiments of the invention, orifice 158 is engaged with just with the treatment catheter 850 soft tip or with its flexible guide wire. Optionally, a guiding catheter 500 with an alternative tip (such as is used with the adrenal vein or the spinal artery) is used to engage the orifice 158 of the ISV 102 and/or orifice 132 of ISV 130. In an embodiment of the invention, the tip is engagement tip 584, described below.

Typically located within ISV 102 (and/or ISV 130) are a series of one-way valves which under nominal operation prevent reflux of blood back down ISV 102 towards testis 104. The valves may be passed (406) by a combination of patient/table positioning, breathing maneuvers, and applied suction via the guiding catheter. If the valves can be easily passed, the treatment catheter 850 is optionally introduced with or without a guide wire in place. Treatment catheter 850 is maneuvered to the lowest desired point for scleropathy (i.e. occlusion), usually just above the inguinal ligament, in accordance with an embodiment of the invention. In an embodiment of the invention, the lowest point desired for scleropathy is selected factoring in a number of factors including risk of reflux into the scrotum and/or availability of secondary veins for adequate rerouting of venous flow. Optionally, the lowest desired point is higher than just above the inguinal ligament.

In an embodiment of the invention, intravenous contrast is injected into the treatment catheter 850 under fluoroscopic control in order to obtain venographic images of the ISV 102, both distally and proximally, using appropriate manipulation of a tilting fluoroscopy table to establish the anatomy of the ISV 102 and its communicating and collateral tributaries. In an embodiment of the invention, contrast agent and/or sclerosant is passed through treatment catheter 850 via a contrast agent and/or sclerosant lumen 866. This is used to optionally map (408) both collateral ISV tributaries as well as interconnectivity between the ISV 102 and other retroperitoneal veins, in an embodiment of the invention.

In some embodiments of the invention, if there is interconnectivity evident to other significant veins, such as the renal capsular vein, the ureteral vein and paravertebral retroperitoneal veins, these vessels, and their interconnections are noted, and it is determined if these interconnected veins can be occluded by treatment of the ISV or need to be separately occluded and/or disconnected from the ISV by using localized scleropathy and/or other occlusive techniques, before proceeding with the general scleropathy occlusion of the ISV.

In some embodiments of the invention, a chemical sclerosant agent is chosen due to its tendency to flow at least some degree down tributary and/or branch veins from the ISV and occlude them, when otherwise the veins might have functioned as bypass conduits. Usage of a chemical sclerosant, therefore, may obviate the need to separately occlude branch and/or tributary and/or connecting veins as it is anticipated that the chemical sclerosant will flow at least some distance into these veins and occlude them, particularly by irritating them enough to cause them to close and stick shut. In some embodiments of the invention, no branch veins are occluded. In some embodiments of the invention, up to a few branches are occluded simultaneously with the ISV. Optionally, several or more branches are occluded simultaneously with the ISV. In some embodiments of the invention, a mechanical plug and/or cement is used to occlude branch veins.

If interconnecting vessels are to be occluded separately (e.g. if it is believed that use of a chemical sclerosant in the ISV will not effectively occlude the interconnecting vessels), this is optionally done before turning to the main ISV 102 and its branches, in an embodiment of the invention. Treatment catheter tip 852 is positioned in the vessel to be occluded and a very small amount of sclerosant mixture (optionally less than 0.5 ml) is applied into the vein. Treatment catheter tip 852 remains in place for a time determined to be sufficient for rendering a desired therapeutic effect; in an embodiment of the invention, 2-4 minutes. After a period of time sufficient for rendering the desired therapeutic effect, the catheter can be removed and a small volume of iodinated contrast material can optionally be injected into the ISV to confirm occlusion of the branch, in accordance with an embodiment of the invention. In an embodiment of the invention, the patient is monitored to ensure that no contrast material flows to the prostate while the patient is upright or standing.

Alternatively, if the interconnecting vein is larger than 2-3 mm, an intravascular microcoil, or other occluding device known in the art, is optionally used to occlude the interconnecting vein. Other techniques for vascular occlusion may be used including, but not limited to, various biologically compatible adhesives, local heating with a radiofrequency driven catheter, heat with a coil, cryoablation, focused ultrasound and/or any methods of sealing blood vessels known in the art.

In an embodiment of the invention, upon conclusion of mapping (408) the relevant anatomy of the patient, treatment commences to occlude at least part of the ISV 102. Scleropathy treatment of ISV 102 includes occluding a length of ISV 102 extending from just above the inguinal ligament to within a few cm of the ISV orifice 158 where ISV 102 connects to the left renal vein 150; thus treatment catheter 850 is positioned at the commencement of therapy just above the inguinal ligament, in some embodiments of the invention. It should be understood that less or more of ISV 102 is occluded depending on the opinion of the attending medical professional and/or the needs of the patient. Furthermore, as described elsewhere herein, other vascular structures besides ISV 102 may need to be occluded in order to achieve therapeutic success according to some embodiments of the invention.

In an embodiment of the invention, occlusion of at least part of ISV 102 is performed by intravenous application of sclerosant in ISV 102 while ISV 102 is compressed (410) in the inguinal region to prevent reflux of sclerosant downwards and/or flushing out of the sclerosant upwards with the venous blood flow. In an embodiment of the invention, an automatic external compression device is placed in the appropriate location on the patient and is activated by an operator at the controller or automatically by the controller itself.

In some embodiments of the invention, physical compression (410) is augmented and/or supplanted by employing other techniques, such as by applying suction to the treatment catheter during injection (414), injection (414) described below. In an embodiment of the invention suction and injection are performed via separate ports in the treatment catheter. In some embodiments of the invention, compression (410) is augmented and/or supplanted by injecting a non-hazardous fluid, such as saline, upstream from the treatment site where the sclerosant is being injected (414) thus creating a pressure gradient across the vein with the direction of flow away from the scrotum. Optionally, the pressure exerted by the non-hazardous fluid is sufficient to prevent reflux but not so strong as to force sclerosant away from the treatment site. In some embodiments of the invention, the patient is placed in a Trendelenburg position, using gravity to assist in avoidance of reflux. In some embodiments of the invention, the manual compression and/or external tourniquet is used to channel the flow of sclerosant.

In an embodiment of the invention, contrast material is injected into treatment catheter 850 after the inguinal region is compressed in order to test the efficacy of the compression and to verify that flow towards the scrotum is restricted. Compression of the inguinal region is adjusted until it can be seen that flow towards the scrotum is largely prevented, in an embodiment of the invention.

Once it is verified (412) that prevention of flow towards the scrotum has been achieved, the sclerosant mixture is slowly injected (414) into ISV 102 via treatment catheter 850 while treatment catheter tip 852 is withdrawn (416) along the predetermined length of ISV 102 to be occluded. In this manner, a single injection of sclerosant accompanied with a smooth, continuous withdrawal movement of treatment catheter 850 along the section of ISV 102 to be occluded provides a “single shot” procedure for performing scleropathy. In some embodiments of the invention, at least one port is located at the distal tip of the treatment catheter for injecting (414) sclerosant and/or aspiration (420), described below, of fluidic substances.

In some embodiments of the invention, treatment catheter 850 is withdrawn a total of about 20-25 cm in ISV 102. Depending on the size of the ISV, a mixture of up to 4 ml sclerosing agent with 0.25-0.5 ml 2% local anesthesia, such as lidocaine, is made and agitated in a 3-5 ml plastic syringe, in some embodiments of the invention. In some embodiments of the invention, the mixture can be transferred from syringe to syringe using a stopcock arrangement to produce a foam prior to injection of the sclerosant into the patient.

In some embodiments of the invention, sclerosant is metered as it is being injected (414) and/or the speed of withdrawal (416) is measured such that treatment can be carried out according to a predefined volume injected to speed of withdrawal ratio. Optionally, position indicators are located on the shaft of the treatment catheter at the proximal end so that the controller and/or the attending medical professional can monitor the indicators as the treatment catheter is withdrawn. In some embodiments of the invention, it is predetermined the amount of sclerosant per vein unit length or diameter traversed is used during treatment. In some embodiments of the invention, treatment catheter 850 is preloaded with a pre-measured dosage of sclerosant and/or a sclerosant mixture, such as described above, for injection (414).

In an embodiment of the invention, withdrawal (416) and/or injection (414) is performed automatically by a device adapted for providing motion and/or other operations (e.g. injecting sclerosant) to the catheters used in the scleropathy procedure. Optionally, the device is operated automatically by a controller. Optionally, an attending medical professional controls the device. In an embodiment of the invention, the controller is programmed with various sclerosant dosages and/or types and the corresponding movement rates which are desired in order to render effective scleropathy treatment.

The injected sclerosant is optionally allowed to remain (418) at the treated area for a period of time to allow for maximal irritant effect. Optionally, 5-10 minutes are allowed to elapse to allow for the sclerosant to take effect. In an embodiment of the invention, the fluoroscopy table on which the patient is positioned is tilted slightly. Optionally, the table is positioned “feet down”. Optionally, the table is rotated up to 10 degrees from horizontal. In an embodiment of the invention, for up to 5-10 minutes after the injection the table is kept in horizontal position. In an embodiment of the invention, the time the table is kept in a horizontal position coincides with the length of the optional pause (418).

After injection (412) of the sclerosant and the optional pause (418) to allow the sclerosant to take effect, the contents of the treated vein are aspirated (420) through the contrast agent and/or sclerosant lumen 866 of the treatment catheter 850 by applying suction and are optionally discarded, in accordance with an embodiment of the invention. Compression of the ISV 102 in the inguinal region is released (422) and treatment catheter 850 is flushed with a small amount of saline and/or intravenous contrast, in an embodiment of the invention. Occlusion of ISV 102 is confirmed (424), in an embodiment of the invention, by injecting a contrasting agent above the occlusion site and observing the treated area. In some embodiments of the invention, confirmation (424) is optional. Optionally, the patient is in a semi-erect position (20-50 degrees) during confirmation (424).

In an embodiment of the invention, if the ISV 102 is not successfully occluded, a second pass of treatment catheter 850 is performed, relocating the tip 852 of the catheter 850 into the segment of the ISV 102 requiring additional treatment. Some or all of the actions performed for the initial injection are repeated until it is determined that the ISV 102 has been occluded. In some embodiments of the invention, confirmation (424) is not performed and if ISV is not successfully occluded and/or if branch veins become enlarged, the procedure is performed again after some time. Optionally, some time is a matter of days, weeks or months.

In an embodiment of the invention, once the ISV 102 is successfully occluded, the guiding catheter 500 is flushed with sterile saline solution. Optionally, a contrast injection is made into the left renal vein 150 as a diagnostic venogram to look for any collateral veins that might fill the ISV 102 that may not have been visible before the ISV 102 had been occluded. In some embodiments of the invention, any collateral veins which appear following occlusion of the ISV 102 are noted for possible occlusion. In an embodiment of the invention, noted veins in the abdominal area are treated and/or occluded (veins in the scrotum are not occluded).

In some embodiments of the invention, the right ISV 130 is also occluded in order to treat the patient's medical condition. A diagnostic venogram is obtained of the right renal vein 152 and appurtenant areas, in an embodiment of the invention. Optionally, guide catheter 500 is used by maneuvering the catheter 500 from the left renal vein 150 to right renal vein 152 and injecting contrast agent through catheter 500. In an embodiment of the invention, the left sided guide catheter 500 is adequate to enter the right renal vein 152 for the venographic demonstration and since it is already inside, it used for gathering the diagnostic venogram. In some embodiments of the invention, a preliminary search for the right ISV 130 is performed with the guide catheter 500 to see if right ISV 130 is in the usual anatomic location. If guide catheter 500 is used to obtain the venogram, it is exchanged for right guide catheter 550, right guide catheter 550 being adapted for use with the right renal vein 152 and/or right ISV 130. Right guide catheter 550 is shown and described in more detail with respect to FIG. 5B.

Due to idiosyncrasies of the right side, it may be difficult to identify the junction of ISV 130 with IVC 106. In fact, there may be multiple junctions between ISV 130 and IVC 106 or in some instances ISV 130 will intersect right renal vein 152 instead of IVC 106. In addition, there may be a valve at the entry orifice 132 to ISV 130 which further increases the difficulty in entering ISV 130 with a catheter. In an embodiment of the invention, the use of table positioning (e.g. “feet up” Trendelenburg position) and/or breathing maneuvers may help. Once right ISV 130 has been entered by guide catheter 550, shown in more detail in FIG. 6B, treatment catheter 850 is advanced into ISV 130 in a similar fashion as performed in ISV 102. In an embodiment of the invention, the remainder of the treatment rendered to ISV 130 is performed in the same fashion as on the left ISV 102. Right side guide catheter 550 is provided with a bulb at its tip 552 to facilitate entering the orifice 132 of right ISV 130 if there is a competent valve present near or at the orifice 132.

At the conclusion of the treatment, when right ISV 130 appears occluded and no collateral filling (“bypass”) of the right ISV 130 is seen, treatment catheter 850 is withdrawn and the guiding catheter 550 is pushed upwards to dislodge it from the right ISV orifice 132. In some embodiments of the invention, guiding catheter 550 is turned and straightened by either maneuvering the tip 552 into the left renal vein 150 or downwards into the orifice of the left internal iliac vein. The guiding catheter 550 and the femoral vein sheath are removed and in some embodiments of the invention, the puncture site is compressed to reduce bleeding and to commence patient recovery.

As discussed elsewhere herein, single shot methodology with treatment catheter 850 can be applied for treatment of varicocele, BPH, some forms of cancer and/or the back flow (reflux) of venous blood, rich in testosterone (relative to normal levels in the blood circulation), from the testes to the prostate. In addition, some actions may be performed more than once and/or are optional depending on the individual patient's needs and/or the opinion of the attending medical professional.

Single Shot Scleropathy with Alternative Catheters and Techniques

FIG. 4B shows a flowchart 450 of a single shot scleropathy procedure using at least one device which, alone or in combination with other devices, proscribes an area in a vein to be treated by occluding two opposing ends of the area. In some embodiments of the invention, a tandem balloon configuration is provided to proscribe the treatment area. It should be understood that a tandem balloon configuration could be provided by a single device or a combination of devices wherein at least a proximal balloon (i.e. balloon near the intersection of the ISV and the IVC/renal vein) and a distal balloon (i.e. balloon located nearer the inguinal region and away from the IVC/renal vein) are provided to proscribe a lumen between them to be treated.

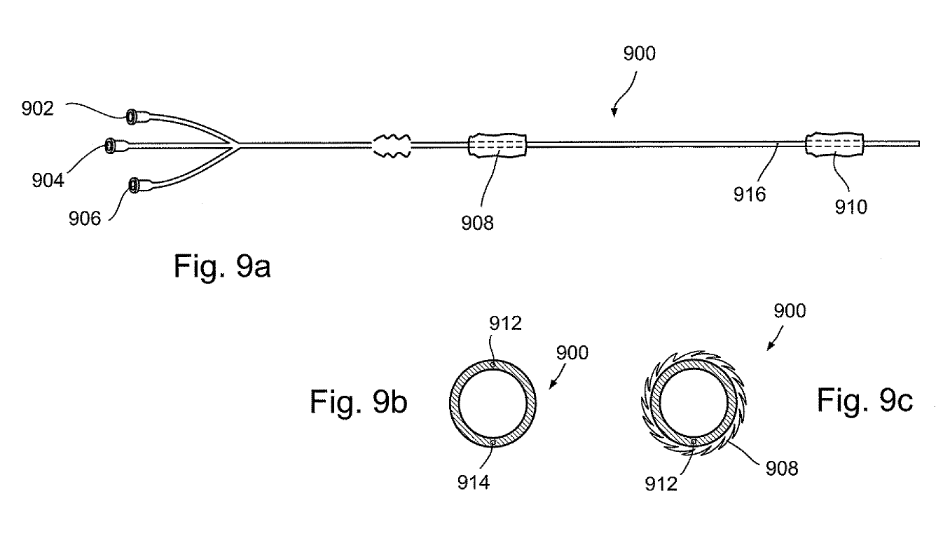

In some embodiments of the invention, a tandem balloon treatment catheter 900 is used to deliver sclerosant to the vasculature being occluded. Tandem balloon treatment catheter 900 is shown in more detail in FIGS. 9A-9C. Optionally, tandem catheter 900 is a 3 F catheter. In an embodiment of the invention, left ISV 102 is treated before right ISV 130. It should be noted however, that right ISV 130 could be treated first instead.

In an embodiment of the invention, the proximal 908 and distal balloons 910 are selectively inflatable and deflatable by using side hole ports with a fluid connection between each balloon's inflation/deflation lumen 912, 914 located within the treatment catheter 900 and the inside area of the balloons 908, 910, as shown in FIG. 9C. In an embodiment of the invention, the balloons 908, 910 are adapted to inflate sufficiently to substantially hinder fluid flow through the vasculature in which the treatment catheter is located. Optionally, the vasculature is an ISV 102, 130. In an embodiment of the invention, when inflated the distal 910 and proximal 908 balloons define a lumen or space therebetween. In an embodiment of the invention, the treatment catheter 900 is adapted to inject sclerosant between the distal 910 and proximal 908 balloons via an injection side hole port 916.

In an embodiment of the invention, the tandem treatment catheter 900 is provided with a contrast agent and/or sclerosant transporting lumen. Optionally, the space between the balloons is aspirated or evacuated through side hole port 916 using the contrast agent and/or sclerosant transporting lumen by applying suction. In some embodiments of the invention, each balloon 908, 910 is provided with its own inflation/deflation lumen 912, 914, shown in FIG. 9B, whereby each balloon 908, 910 can be individually inflated and/or deflated. Optionally, the tandem treatment catheter 900 is provided with a guide wire lumen through which a guide wire passes when the treatment catheter is being moved to the treatment site. Optionally, the guide wire is retracted from the lumen when the tandem treatment catheter 900 reaches its proper position for treatment.

In some embodiments of the invention where there the treatment catheter is provided with a distal balloon, for example distal balloon 910, the scleropathy procedure starts substantially similar to preparing (402) to passing (406) depicted in flowchart 400. In an embodiment of the invention, at the outset of optional mapping (460), distal balloon 910 is gently inflated (458) to approximate the walls of the ISV 102. Distal balloon 910 is used to occlude the vasculature being treated below the treatment area to prevent reflux of material back towards the scrotum, in some embodiments of the invention. Optionally, distal balloon 910 is inflated under fluoroscopic observation after an iodinated contrast has been injected to opacify the ISV 102. Optionally, the distal balloon 910 is inflated using dilute iodinated contrast material (1 ml contrast to 3-5 ml sterile saline ratio). In some embodiments of the invention, at least one side hole port 914 is provided to treatment catheter 900 for injection of substances into distal balloon 910 (e.g. to inflate distal balloon 910) and at least one side hole port 916 is provided for injection of contrast agent and/or sclerosant into the vasculature to be occluded.

In an embodiment of the invention, compression (462) is applied to the vasculature being occluded, for example the ISV, and after inflation of distal balloon 910 the remaining blood in the ISV, above the distal balloon 910 (extending towards the renal vein) is aspirated, as best as reasonably possible. In an embodiment of the invention, the inflated distal balloon 910 is sufficient to occlude the ISV. Optionally, manual compression (462) is maintained to prevent reflux of any sclerosant into the scrotum. In order to test the efficacy of the occlusion provided by distal balloon 910, a small of contrast is optionally injected via at least one side hole port 916 into the lumen of the ISV, above the occlusion site. The injection of intravenous contrast is used to confirm (464) that there is no reflux of contrast material into the veins below the inguinal region (towards the scrotum), confirming thorough isolation of the ISV below the site of the distal balloon 910.

If it is determined that the ISV is satisfactorily occluded by distal balloon 910, then the distal balloon 910 is repositioned and/or re-inflated and/or digital compression of the groin is adjusted until there is no contrast which passes the inguinal region, in an embodiment of the invention.

In an embodiment of the invention, a proximal balloon 908 of the treatment catheter 900 is inflated (466) to occlude the vasculature being treated creating between the two inflated balloons 910, 908 a lumen within the vein which is substantially, if not completely, isolated from fluid flow. In an embodiment of the invention, the lumen defined by the two inflated balloons 910, 908 represents the segment of the vein to be occluded. Following confirmation of the inflation of the proximal 908 and distal 910 balloons, the side hole port 916 of the tandem balloon catheter 900 is used to aspirate (468) the vessel lumen until the lumen is as reasonably empty as possible, in an embodiment of the invention. Sclerosant is injected (470) into the lumen to be treated via at least one side hole port 916, in an embodiment of the invention. In an embodiment of the invention, the volume of sclerosant that is injected into the lumen is tailored to the needs of the patient and/or the individual characteristics of the sclerosant and/or sclerosant mixture and/or being used. Optionally, 3-4 ml of sclerosant mixture is injected into the lumen.

After an optional pause (472) to allow the sclerosant to take effect, the balloons 910, 908 are deflated (474) and the contents of the treated vein are aspirated (476) through the catheter 900, in an embodiment of the invention. The optional pause lasts from five to ten minutes after the application of the sclerosant mixture; however the pause can be tailored to characteristics of the particular sclerosant being used. In an embodiment of the invention, compression of the inguinal region is removed and/or deactivated (478).

Treatment catheter 900 is flushed with a small amount of saline and/or intravenous contrast, in an embodiment of the invention. Occlusion of the vein is optionally confirmed (480), in an embodiment of the invention, by injecting a contrasting agent above the occlusion site and observing the treated area. Optionally, the patient is in a semi-erect position (20-50 degrees) during confirmation (480). Naturally, if the treated vein is occluded contrast agent will not directly traverse past the occluded area (it is possible however, that via branch veins there is flow past the occlusion indirectly).

In an embodiment of the invention, if the vein is not occluded, a second pass of the treatment catheter 900 is done, relocating the tip of the catheter into the distal pelvic portion of the ISV 102 under fluoroscopy, aspirating the contents of the vein through the treatment catheter 900, and then re-inflating the distal 910 and proximal 908 balloons of the treatment catheter 900 as described above. Injection is repeated in order to effect occlusion of the ISV.

However, if the ISV 102 is partially occluded after the first or subsequent treatment and the treatment catheter 900 can only be partially re-extended, in an embodiment of the invention, one or both of the balloons 910, 908 are deflated and treatment catheter 900 is operated similarly to treatment catheter 850 for positioning and/or injection. Treatment is continued until it is determined that the vein being treated is successfully occluded.

In some embodiments of the invention, confirmation (480) is not performed and if ISV is not successfully occluded and/or is partially occluded and/or branch veins become enlarged, the procedure is performed again after some time. Optionally, some time is a matter of days, weeks or months.

Upon attainment of occlusion of the vein being treated, treatment catheter 900 is removed, in an embodiment of the invention. The guiding catheter 500 is flushed with sterile saline solution and a semi-erect contrast injection is made into the left renal vein 150 to look for any collateral veins that fill the ISV 102 that may not have been visible before the main ISV 102 had been occluded. When it is determined that there is no filling of the left ISV 102 directly or indirectly, then the right ISV 130 is occluded following the same basic techniques as described elsewhere herein with respect to right ISV 130 but taking into account that tandem balloon treatment catheter 900 is being used.

In an embodiment of the invention, a treatment catheter 800 provided with a single distal balloon 810 is used to perform a single shot treatment of a vein. An exemplary treatment catheter 800 is shown in FIGS. 8A-8C. A distinction between using treatment catheter 900 and treatment catheter 800 is that instead of filling an enclosed lumen with sclerosant as with treatment catheter 900, treatment catheter 800 ejects sclerosant into the vein to be treated as it is withdrawn towards the renal vein, similar to treatment catheter 850. Optionally, distal balloon 810 is located up to 1-2 cm from a distal end 820 of the catheter 800. In an embodiment of the invention, distal balloon 810 is used to open and push aside valves 108 in the ISV as the catheter 850 is being withdrawn. In some embodiments of the invention, some or all valves 108 are sealed by the scleropathy. In some embodiments of the invention, some or all valves 108 are undamaged.

In an embodiment of the invention, the distal balloon 810 is selectively inflatable and deflatable by using a side hole port 818 with a fluid connection between a balloon inflation/deflation lumen 812 located within the treatment catheter 800 and the inside area of the balloon. In an embodiment of the invention, air for inflation and/or deflation passes through air input/output port 802. In an embodiment of the invention, the distal balloon 810 is adapted to inflate sufficiently to substantially hinder fluid flow through the vasculature in which the treatment catheter 800 is located. Optionally, the vasculature is an ISV. In some embodiments of the invention, the treatment catheter 800 is adapted for use in a single shot scleropathy procedure wherein the treatment catheter 800 can inject sclerosant and be withdrawn with the balloon 810 inflated simultaneously.

As shown in FIG. 8B, treatment catheter 800 is also provided with a contrast agent and/or sclerosant lumen 816 through which contrast agent and/or sclerosant are fed through the treatment catheter 800 to a treatment site within the vasculature, in some embodiments of the invention. In an embodiment of the invention, contrast agent and/or sclerosant pass through contrast agent and/or sclerosant input/output port 804. In an embodiment of the invention, the treatment catheter 800 is adapted to inject contrast agent and/or sclerosant on the proximal side of the distal balloon 810 (i.e. downstream from the balloon 810). Optionally, aspiration/evacuation of the treatment site is performed using the contrast agent and/or sclerosant lumen 816 by applying suction to the lumen.

In an embodiment of the invention, the treatment catheter is provided with a guide wire lumen 814 through which a guide wire passes when the treatment catheter 800 is being moved to the treatment site. Optionally, the guide wire is retracted from the lumen 814 when the treatment catheter 800 reaches its proper position for treatment. In an embodiment of the invention, the guide wire passes in and/or out of guide wire input/output port 806.

In some embodiments of the invention, the distal balloon fitted treatment catheter 800 is adapted to perform a valve breakthrough procedure by inflating the balloon 810 just downstream of the valve 108 creating a vacuum effect, opening the valve 108, and enabling the transit of the treatment catheter 800 therethrough. In an embodiment of the invention, the balloon 810 is deflated after the valve 108 has been passed through.

Optionally used with treatment catheter 800 or treatment catheter 850 is a balloon tipped guide catheter 570, 580, examples of left and right catheters are shown in FIGS. 5C and 5D, respectively. Using an embodiment of the invention as an example where balloon tipped left side guide catheter 570 and treatment catheter 800 are used, a balloon 572 of balloon tipped guide catheter 570 is inflated (466) and used to approximate the function of proximal balloon 908 of treatment catheter 900 during treatment of the vein.

In an embodiment of the invention, balloon 572 of balloon tipped guide catheter 570 is located approximately 4-8 mm from the tip 574 of guide catheter 570. In some embodiments of the invention, the balloon 572, when inflated, has a 3-5 mm diameter, although varying sizes are optionally used depending on the treatment site conditions. Optionally, the balloon 572 is 3-5 mm “long”, extending along catheter 570 near its tip 574 for 3-5 mm. In some embodiments of the invention, overall lengths and angles of curvature of balloon tipped guide catheter 570 are similar to those of non-balloon tipped guide catheter 500.

In an embodiment of the invention where balloon tipped guide catheter 570 is used with regular treatment catheter 850, then the balloon is used to occlude the ISV while the treatment catheter 850 is drawn towards it. At the conclusion of sclerosant injection, aspiration (420) of remaining blood and sclerosant is performed by via the treatment catheter 850. In some embodiments of the invention, a left side guide catheter 500 is adapted to be balloon tipped. In some embodiments of the invention, a right side guide 550 catheter is adapted to be balloon tipped. In some embodiments of the invention, guide catheter 570 is 4-6 French. In some embodiments of the invention, balloon 572 inflates to 3-6 mm diameter, extending 1-3 cm along guide catheter 570 up to 1-2 cm from the tip 574 of guide catheter 570.

It should be noted that a similar method of use and/or function of right sided balloon tipped guide catheter 580 and its appurtenant balloon 582 is envisioned, in some embodiments of the invention. Primary distinctions between left guide catheter 570 and right guide catheter 580 being the shape of the catheters (each one is pre-shaped to interface with the anatomy on each catheter's respective side) and that in some embodiments of the invention, right side guide catheter 580 (and optionally right side guide catheter 550) is adapted to provide an engagement tip 584 for “latching” guide catheter 580 onto right ISV 130 at the right ISV orifice 132. In an embodiment of the invention, engagement tip 584 is asymmetrical, since in some embodiments of the invention a bulge is needed on the upper side of the tip to engage the wall of the IVC. Optionally, engagement tip 584 is comprised of a bulb, ovoid or a ball. Optionally, engagement tip 584 includes a protrusion such as a hook and/or barb. In some embodiments of the invention, engagement tip 584 is 1 mm to several mm in length. In some embodiments of the invention, engagement tip 584 is 0.5 mm-2 mm in diameter. Optionally, engagement tip 584 is rigid. Optionally, engagement tip 584 is flexible. In some embodiments of the invention, overall lengths and angles of curvature of balloon tipped guide catheter 580 are similar to those of non-balloon tipped guide catheter 550, described elsewhere herein.

In an embodiment of the invention, engagement tip 584 includes a balloon 582. In an embodiment of the invention where balloon 582 is used for engagement, it is inflated to approximately 3 mm-4 mm near the orifice 132. In use, guide catheter 580 is pushed gently upwards (with the balloon inflated) towards to engage the upper wall of orifice 132, thus widening the aperture and also opening the valve 108 at orifice 132, allowing the treatment catheter to be inserted into the ISV. The balloon is then deflated and the guide catheter 580 is advanced into the orifice 132, stabilizing the devices and allowing proximal control on the blood flow.

In some embodiments of the invention, a telescoping treatment catheter is used for performing scleropathy. The telescoping catheter, in an embodiment of the invention, is not advanced towards the treatment site like catheters 800, 850, 900 by pushing the catheter down ISV. Instead, telescoping catheter is placed in the ISV, but not as far as the treatment site, with a distal portion of the catheter in a folded state. Upon application of internal pressure in the catheter, for example using a fluid, the distal folded portion deploys into the vessel being treated and at least as far down in the vessel as the treatment site. Optionally, the fluid used to deploy the distal folding portion is the sclerosant being used to treat the vessel. Optionally, the fluid is saline. In an embodiment of the invention, the folded portion when deployed is up to 25 cm long. Optionally, the folded portion when deployed is up to 10 cm long. In an embodiment of the invention, the folded portion when deployed is up to 6 mm in diameter. In an embodiment of the invention, at least one channel is provided in telescoping channel for fluid flow. Optionally, the channel is located in the folded material of the distal folded portion and when fluid is applied through the telescoping catheter the fluid acts to unfold the distal folded portion.

As discussed elsewhere herein, single shot methodology with treatment catheters 800, 900 can be applied for treatment of varicocele, BPH, some forms of cancer and/or the back flow (reflux) of venous blood, rich in testosterone (relative to normal levels in the blood circulation), from the testes to the prostate. In addition, some actions may be performed more than once and/or are optional depending on the individual patient's needs and/or the opinion of the attending medical professional.

Single Shot Scleropathy with Alternative Access Points

In some embodiments of the invention, access to ISVs 102, 130 is achieved by entering the vasculature without using the femoral vein. For example, access is obtained by entering an arm vein, similar to a PICC line insertion. Alternatively, vascular access is obtained using the right internal jugular vein.

In some embodiments of the invention, performing scleropathy using jugular or arm access is achieved using a single guide catheter in combination with a treatment catheter regardless of which ISV (i.e. left or right) is being treated. Additionally, alternatively and/or optionally, scleropathy is performed using a guide wire to direct the treatment catheter into the treatment area and/or ISV. In some embodiments of the invention, the treatment catheter is adapted to be used without a guide wire or a guide catheter. In an embodiment of the invention, the treatment catheter is 4 F. Optionally, the treatment catheter is a “Head Hunter” catheter. Optionally, the treatment catheter is 100-120 cm long. In some embodiments of the invention, the treatment catheter is provided with a hydrophilic tip.

In an embodiment of the invention where a guide catheter is used in combination with a treatment catheter, the guide catheter is optionally provided with an inflatable balloon (similar to the balloon 572 provided to balloon tipped guide catheter 570) to permit occlusion of the upper ISV orifice while the sclerosant is injected through the treatment catheter and into the area being treated. Optionally, the treatment catheter is provided with a distal balloon (i.e. located near the tip of the catheter away from the renal vein) and is used in combination with a balloon tipped guide catheter to provide operation similar to tandem balloon treatment catheter 900, wherein a side hole port located between the distal balloon and the guide catheter injects sclerosant into a lumen formed by the distal balloon and the balloon on the guide catheter.

In an embodiment of the invention which does not use the coaxial system (treatment catheter within the guide catheter), the treatment catheter is provided with a proximal occlusion balloon about 20-25 cm above its tip (i.e. located near the intersection of the ISV and the renal vein/IVC) to occlude the lumen of the ISV being treated. Optionally, a second occlusion balloon is provided to the distal portion of the to treatment catheter located away from the renal vein/IVC junction. The sclerosant is injected via a side hole port located in a portion of the treatment catheter between the two balloons, if there are two, or 3-5 cm from its distal end. The scleropathy is performed at the treatment site depending on the device configuration as described elsewhere herein. In an embodiment of the invention, the process is repeated at a treatment site within the patient's other ISV.

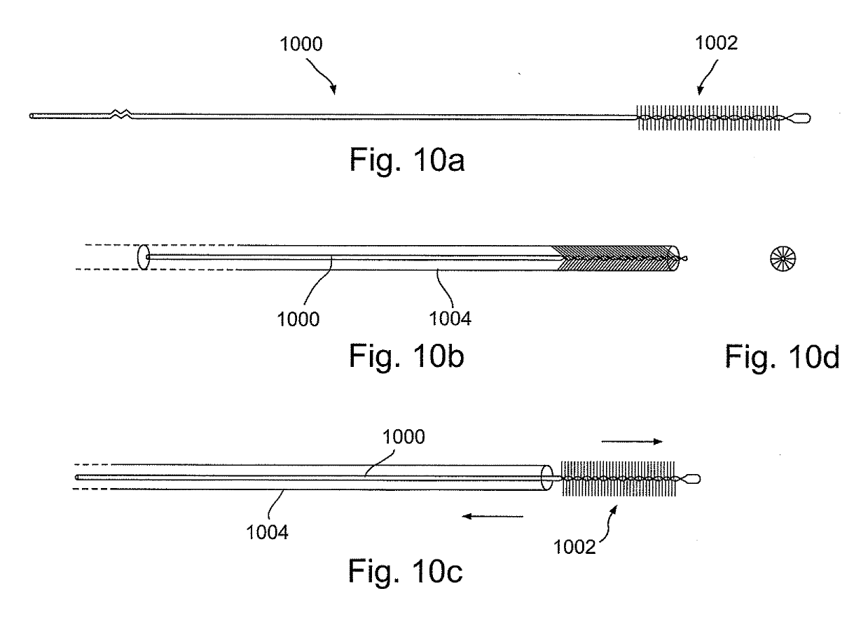

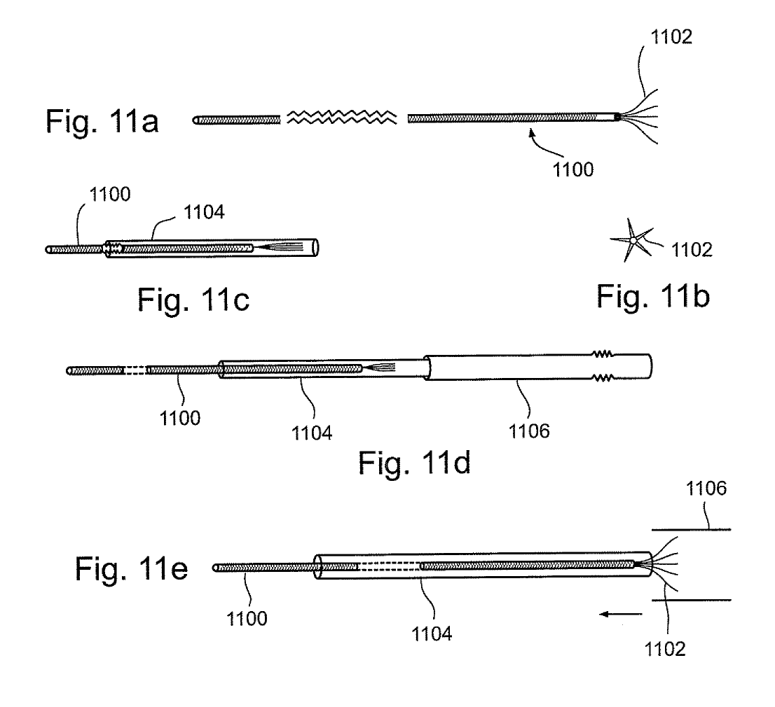

It should also be noted that alternative “irritants” for achieving vascular occlusion are capable of use with the methods described herein, or adapted versions thereof, and that application of one technology over another is left to the professional judgment of the doctor and/or the needs of the patient. Examples of mechanical irritant devices are shown and described with reference to FIGS. 10A-11E, below.

### Expedited Vein Access Procedure

FIG. 7 shows a flowchart 700 of an expedited vein access procedure, in accordance with an exemplary embodiment of the invention. Conventionally, accessing certain veins with treatment devices, for example the left and right ISVs, in particular the right ISV, is time consuming and requires high skill on the part of the attending medical professional. In an embodiment of the invention, vein access is achieved faster and easier using pre-shaped devices and the valve traversal methodology described herein. It should be noted that a synergy exists between certain methods and device features described herein since these common device features (e.g. distal balloon) are used for the valve traversal method and for the single shot methods described elsewhere herein.

In an embodiment of the invention, left sided guide catheter 500 is adapted to interface with left renal vein 150 and/or left ISV 102 in a typical anatomic configuration, as seen in FIG. 6A. For example, the main shaft of the guide catheter 500 is pre-shaped to make an approximate 90-130 degree turn (i.e. a first curve), approximating the angle of entry to the IVC 106 by the renal vein 150, and then makes another turn (i.e. a second curve) approximately 100-125 degrees approximating the angle of incidence of the left ISV 102 to the left renal vein 150, wherein the two turns of the guide catheter 500, along with its flexible nature, allow guide catheter 500 to provide relatively quick ISV access to the attending medical professional. In an embodiment of the invention, the overall guide catheter 500 length is about between 600 mm and 700 mm. In some embodiments of the invention, the distance between the two turns is approximately 30 mm.

In some embodiments of the invention, right sided guide catheter 550 is adapted to interface with right ISV 130 directly (without passing through right renal vein 152) because in most patients, the right ISV 130 will intersect with the IVC 106 and not with the right renal vein 152. An adaptation of right sided guide catheter 550 for interfacing with right ISV 130 includes providing a bulb at its tip 552 to facilitate entering the orifice 132 and/or engagement of right ISV 130 if there is a competent valve 108 near or at the orifice 132. In an embodiment of the invention, guide catheter 550 is pre-shaped to be first curved at least 170 degrees and up to 180 degrees (with a small, second curve in the opposite direction at the tip, to approximate the angle of incidence of the right ISV 130 to the IVC 106, near the tip 552), as shown in FIG. 6B. In an embodiment of the invention, guide catheter 550 is between 600 mm and 700 mm long. In some embodiments of the invention, approximately 6 cm of catheter 550 is after the first curve. In an embodiment of the invention, the engagement tip 552 is used to provide a stable entrance to ISV 130, by interfacing or mating with a small groove or recess in the orifice 132, as well as possibly traversing the first valve 108 of the ISV 130 (which in many patients is located near the orifice 132).

It should be recognized that the lengths and angles described above are by way of example only and that the actual configurations can vary depending on a number of factors, including the individual needs of the patient and commercial considerations.

Pre-shaped guide catheters, for example guide catheters 500, 550 described above, are inserted (702) into the patient's body at an access point such as the femoral vein. In an embodiment of the invention, the flexible guide catheter is inserted (702) in a substantially straight configuration even though its preconfigured shape may not be straight. The guide catheter opens into its pre-defined shape once it has enough room to expand, for example upon entrance to the IVC 130. The guide catheter is maneuvered (704) to the IVC 106 on the way to an ISV 102, 130 where the guide catheter expands to its preconfigured shape, in an embodiment of the invention. Once in the IVC 106, the medical professional interfaces (706) the guide catheter with the anatomical feature that the guide catheter was pre-shaped to mate with, in an embodiment of the invention.

In an embodiment of the invention, a treatment catheter (e.g. 800, 850, 900) is inserted (708) into the guide catheter and advanced through the guide catheter to the first valve 108 of the ISV 102, 130 being targeted. The treatment catheter, if provided with a distal balloon, inflates (710) the balloon just downstream of the valve 108 being traversed. The expansion of the balloon and subsequent blockage of the vessel creates a vacuum effect just down stream of the valve 108, causing the valve to open. In an embodiment of the invention wherein the treatment catheter is not provided with a distal balloon, a balloon fitted guide catheter is optionally used to enable a quick traversal of the first valve 108. With the valve 108 still open, the treatment catheter is advanced (712) past the valve, in an embodiment of the invention. The balloon is deflated (714) once the valve 108 has been traversed by the treatment catheter, in an embodiment of the invention. In some embodiments of the invention, the balloon remains inflated if the treatment catheter has reached the treatment site. Assuming the treatment catheter has not reached the treatment site after the traversal, the treatment catheter is advanced (716) further until the next valve is reached at which time the traversal process is repeated (718). As described above, the process continues until the treatment catheter has reached the treatment site and/or until there are no more valves 108 to traverse.

### Exemplary Mechanical Irritation Devices

Referring to FIG. 10A, a side view of a brush irritation device 1000 is shown, in accordance with en exemplary embodiment of the invention. Brush irritation device 1000 includes a flexible shaft similar to a flexible, intravascular guide wire. In an embodiment of the invention, the shaft is 140 cm long. Optionally, the shaft has an outer diameter of 0.0534 cm (corresponding to 0.021 in). At the operational end of irritation device 1000, is a brush section 1002 welded onto the shaft. In an embodiment of the invention, irritation device 1000 is adapted for operation in venous plexus spaces and/or other small vein areas and/or hard to reach veins. In an embodiment of the invention, brush section 1002 is comprised of fine bristles fixed into a finely twisted wire, thus resembling a pipe cleaner. Optionally, the bristles are nylon. In an embodiment of the invention, the brush section 1002 is approximately 1 cm long. In an embodiment of the invention, the effective diameter of the brush is variable to match the inner diameter of the vessel wall being treated. Optionally, the diameter is variable from about 0.3 cm to 0.5 cm since the bristles are flexible.

In an embodiment of the invention, brush irritation device 1000 is loaded into a delivery catheter 1004, shown in FIG. 10B, for transit to the treatment site. Brush section 1002 does not protrude out of delivery catheter 1004 prior to device 1000 deployment, in an embodiment of the invention. A cross sectional view of irritation device 1000 across the brush section 1002 and including the delivery catheter 1004 is shown in FIG. 10D, in accordance with an exemplary embodiment of the invention.

In operation, the distal end of the delivery catheter 1004 is positioned beyond the desired site of treatment. While the delivery catheter 1004 is withdrawn, the brush irritation device 1000 is advanced relative to the delivery catheter 1004 such that the brush section 1002 is deployed out of delivery catheter 1004, shown in FIG. 10C. Once deployed outside of the delivery catheter 1004, the entire assembly 1000, 1002, 1004 is withdrawn along the length of the vein which is to be treated by irritation. The irritation device 1000 is then withdrawn back into the delivery catheter 1004 and is optionally withdrawn completely or a second segment can be treated with the irritation device 1000 in the same fashion as described above.

Prior to introducing sclerosant for treatment of the vein, a diagnostic venogram is obtained in the usual fashion with iodinated contrast material injected under fluoroscopic control into the segment of the vein that has been subjected to the irritation device to confirm that there has not been a breach of the venous wall, in an embodiment of the invention. If a breach is detected, that segment of vein is not treated with sclerosant.

Referring to FIG. 11A, a side view of an arm assembly irritation device 1100 is shown, in accordance with an exemplary embodiment of the invention. Arm assembly irritation device 1100 includes a flexible shaft similar to a flexible, intravascular guide wire. In an embodiment of the invention, the shaft is 140 cm long. Optionally, the shaft has an outer diameter of 0.0534 cm (corresponding to 0.021 in). At the distal end of the shaft are attached a plurality of arms 1102. In an embodiment of the invention, irritation device 1100 is adapted for operation in venous plexus spaces and/or other small vein areas and/or hard to reach veins. In an embodiment of the invention, 5 arms are used, although more or less are optionally used. In an embodiment of the invention, the arms are slightly curved in towards the central, longitudinal axis of the device 1100. In some embodiments of the invention, arms 1102 are symmetrically distributed over 360 degrees around the radius of the shaft, as shown in FIG. 11B. In an embodiment of the invention, the arms 1102 are 6 mm long. The maximum diameter of the arms 1102 is adapted to approximate the diameter of the vessel being treated such that the tips of the arms 1102 abrade the inner wall of the vessel. Optionally, the diameter of the arms 1102 is between 0.4 cm and 0.6 cm. In an embodiment of the invention, the tips of the arms 1102 are configured so that they are not likely to pierce the wall of the vessel being treated, for example the tips are rounded.

In an embodiment of the invention, arm assembly irritation device 1100 is placed in a flexible delivery catheter 1104, shown in FIG. 11C. Optionally, the delivery catheter 1104 is plastic. Referring to FIG. 11D, delivery catheter 1104, with irritation device 1100 inside, is advanced to just past the treatment site in the vein 1106, in an embodiment of the invention. Optionally, the delivery catheter is advanced just distal of the treatment site and then irritation device 1100 is advanced within the delivery catheter 1104 to near the distal end of the delivery catheter 1104, but not so far as to be projecting out of the delivery catheter 1104. In some embodiments of the invention, a preliminary venogram of the segment of the vein to be treated is performed to evaluate the approximate diameter of the vein. Iodinated contrast material is placed into the lumen of the segment of the vein to be treated to opacify the lumen, in an embodiment of the invention. Optionally, movement of the irritation device 1100 within delivery catheter 1104 is with fluoroscopic guidance.

In an embodiment of the invention, delivery catheter 1104 is withdrawn as shown in FIG. 11E, allowing arms 1102 to deploy inside the vessel 1106 being treated. In an embodiment of the invention, delivery catheter 1104 is withdrawn only enough for the arms 1102 deploy to the approximate diameter of the vein being treated (the diameter being approximated by the venogram described above). Once arms 1102 are deployed to the approximate inner diameter of vessel 1106, irritation device 1100 and delivery catheter 1104 are withdrawn together along the segment of vessel being treated, in an embodiment of the invention. Upon traversal of the segment being treated, irritation device 1100 is withdrawn while delivery catheter 1104 is held steady until irritation device 1100 is fully within delivery catheter 1104. In an embodiment of the invention, a diagnostic venogram of the treated segment is performed to confirm that there has not been a breach of the vessel 1106 wall. If the wall has been breached, sclerosant is not injected into this segment of the vein. If no breach is detected, scleropathy is optionally performed according to previously described methods.

### An Exemplary Scleropathy Kit

In an embodiment of the invention, a kit for performing the methods described herein is assembled and/or is sold as a commercial unit. For example, the kit optionally includes some or all of at least one treatment catheter (e.g. 800, 850, 900), at least one guide catheter (e.g. 500, 550, 570, 580), at least one vein sheath, a waste receptacle, sclerosant material and/or materials necessary to create sclerosant mixtures, a tourniquet, at least one guide wire, contrast agent materials and/or instructions for use of the devices and/or materials in the kit. In an embodiment of the invention, the contents of the kit are sterilized.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible to limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing and/or reversing the progression of a condition, substantially ameliorating clinical and/or aesthetical symptoms of a condition and/or substantially preventing and/or delaying the appearance of clinical and/or aesthetical symptoms of a condition.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

It is expected that during the life of a patent maturing from this application many relevant advances in scleropathy and/or treatment of varicocele, BPH and related medical conditions will be developed and the scope of the terms used herein (e.g. catheter, sclerosant, irritant) are intended to include all such new technologies a priori.

1. A method of sealing a network of veins which provide drainage to the testes, comprising:
(a) inserting a treatment catheter into a main vein of said network; and,
(b) causing said main vein to seal over at least a length with a single application of vein irritant such that the network of veins is sealed.

2. A method according to claim 1, wherein said single application comprises pulling back said catheter while applying a sclerotic agent.

3. A method according to claim 1, wherein a distal balloon provided to the catheter is inflated prior to the application of vein irritant.

4. A method according to claim 3, wherein the efficacy of the vein irritant is increased by the distal balloon at least partly hindering flow in the main vein.

5. A method according to claim 1, wherein said single application comprises injecting a sclerotic agent into a lumen which proscribes the length to be sealed.

6. A method according to claim 5, wherein a distal balloon and a proximal balloon are inflated, one on each end of the length, thus defining the lumen.

7. A method according to claim 6, wherein the distal balloon and the proximal balloon are inflated using the treatment catheter.

8. A method according to claim 6, wherein the distal balloon is inflated using the treatment catheter and the proximal balloon is inflated using a guide catheter.

9. A method according to claim 1, wherein the catheter is used to map the network of veins by injecting a contrast agent into the network from the catheter.

10. A method according to claim 9, wherein the length is chosen for sealing based on the map of the network of veins.

11. A method according to claim 1, wherein sealing the network is used for treating at least one of BPH, varicocele, cancer, and the reflux of venous blood from the testes to the prostate.

12. (canceled)

13. A treatment catheter adapted for performing scleropathy, comprising:
a shaft defining a longitudinal axis of the catheter;
a selectively inflatable and deflatable distal balloon located on the shaft;
a side hole port in fluid communication with the distal balloon and a balloon inflation/deflation lumen for selectively inflating and deflating the distal balloon;
a side hole port in fluid communication with a contrast agent and sclerosant lumen and a lumen of a vein being treated, wherein the side hole port is adapted to inject the contrast agent and the sclerosant downstream in the lumen from the distal balloon.

14. A treatment catheter according to claim 13, wherein aspiration of the lumen of the vein being treated is achieved by applying suction through the contrast agent and sclerosant lumen.

15. A treatment catheter according to claim 13, further comprising a guide wire lumen adapted for transit of a guide wire therethrough.

16. A treatment catheter according to claim 13, further comprising a selectively inflatable and deflatable proximal balloon, wherein the distal and proximal balloons define the lumen of the vein being treated therebetween.

17. A treatment catheter according to claim 16, wherein the treatment catheter is adapted to inject sclerosant into the lumen of the vein being treated between the proximal and distal balloons.

18. A treatment catheter according to claim 1, further comprising an engagement tip located at a distal end of the shaft and adapted for engaging an orifice of an ISV.

19. A treatment catheter adapted for performing scleropathy, comprising:
a selectively inflatable and deflatable proximal balloon; and,
a selectively inflatable and deflatable distal balloon,
wherein the proximal balloon and the distal balloon proscribe a lumen therebetween to be treated by scleropathy.

20. A treatment catheter according to claim 19, further comprising dedicated inflation/deflation lumens for the proximal balloon and the distal balloon.

21. A treatment catheter according to claim 20, further comprising side hole ports in fluid communication with each dedicated inflation/deflation lumen and the proximal balloon and the distal balloon.

22. A treatment catheter according to claim 19, wherein at least one balloon is adapted to substantially restrict flow in the lumen.

23. A treatment catheter according to claim 19, wherein the lumen is of an ISV.

24. A treatment catheter according to claim 19, wherein the treatment catheter is adapted to inject sclerosant into the lumen between the proximal and distal balloons.

25. A treatment catheter according to claim 19, further comprising a guide wire lumen adapted for transit of a guide wire therethrough.

26. A treatment catheter according to claim 19, further comprising a contrast agent and sclerosant transporting lumen.

27. A treatment catheter according to claim 26, wherein aspiration of the lumen of the vein being treated by scleropathy is achieved by applying suction through the contrast agent and sclerosant lumen.

28. A treatment catheter according to claim 19, further comprising an engagement tip located at a distal end of the shaft and adapted for engaging an orifice of an ISV.

29-48. (canceled)

49. A method for gaining expedited vein access to veins which drain the testes, comprising:
inserting a pre-shaped guide catheter into an access point;
maneuvering the guide catheter into position opposite an anatomical feature of interest;
interfacing the guide catheter with the anatomical feature;
inserting a treatment catheter into the guide catheter and advancing the treatment catheter to a first valve of the anatomical feature;
inflating a distal balloon of the treatment catheter just downstream of the first valve thereby creating a vacuum effect on the valve causing the valve to open;
advancing the treatment catheter past the valve; and,
deflating the balloon after traversal of the valve.

50. A method of claim 49, further comprising advancing the treatment catheter towards a treatment site.

51-65. (canceled)

66. A method for treating varicocele, comprising:
(a) determining that a patient has incompetent valves in a venous plexus;
(b) inserting a catheter into the venous plexus;
(c) sealing the venous plexus using a single injection of sclerosant which reduces venous pressure on a testis using the catheter.

67. A method according to claim 66, further comprising (d) confirming sealing of the venous plexus by injecting a contrast agent into the venous plexus and observing fluid flow therein.

68-70. (canceled)

71. A method of sealing a network of veins which provide drainage to the testes, comprising:
(a) inserting a treatment catheter into a main vein of said network; and,
(b) causing said vein to seal over at least a length using a plurality of applications of vein irritant without changing direction of a withdrawal motion of the treatment catheter.

72. A method according to claim 71, further comprising stopping after a valve in the vein and just prior to at least one of the plurality of applications.

73. A method according to claim 72, wherein the withdrawal motion instigates a flattening of the valve as the catheter passes through the valve.

74-78. (canceled)

79. A method according to claim 1, wherein no forward movement of the treatment catheter is required.
